(19) United States
(12) Patent Application Publication
(10) Pub. No.: US 2004/0234504 A1
(43) Pub. Date: Nov. 25, 2004

(54) Methods of inhibiting gene expression by RNA interference

(76) Inventors: Inder M. Verma, La Jolla, CA (US); Gustavo Tiscornia, San Diego, CA (US); Oded Singer, San Diego, CA (US)

Correspondence Address: Cathryn Campbell, McDERMOTT, WILL & EMERY, Suite 700, 4370 La Jolla Village Drive, San Diego, CA, 92122, US

(21) Appl. No.: 10/742,740

(22) Filed: Dec. 18, 2003

Related U.S. Application Data
(60) Provisional application No. 60/434,523, filed on Dec. 18, 2002.

(51) Int. Cl. A61K 48/00; C12N 15/867

(52) U.S. Cl. 424/93.2; 435/456

(57) ABSTRACT

The invention provides a lentiviral vector capable of inhibiting the expression of at least one target gene. A lentiviral vector of the invention encompasses a first nucleic acid sequence derived from a target gene transcript and a second nucleic acid sequence corresponding to the reverse complement of said first nucleic acid sequence. A lentiviral vector of the invention capable of inhibiting the expression of a target gene is useful in therapeutic applications to inactivate disease-associated transcripts and thereby reduce the severity of inherited metabolic, infectious or malignant conditions. Methods for inhibiting one or more target genes in a cell as well as methods for producing a non-human mammal in which the expression of one or more target genes is inhibited also are provided by the present invention.

This application is based on, and claims the benefit of, U.S. Provisional Application No. 60/434,523, filed Dec. 18, 2002, and entitled METHODS OF INHIBITING GENE EXPRESSION BY RNA INTERFERENCE, and which is incorporated herein by reference.

This invention was made with government support under grant number 5R01 HL53670 awarded by the National Institutes of Health. The United States Government has certain rights in this invention.

### BACKGROUND OF THE INVENTION

This invention relates to methods for studying mammalian gene function and, more specifically, to methods for inhibiting the expression of a desired gene product by stable expression of short interfering RNAs (siRNAs) using lentiviral vectors.

Mammalian genetic studies have been hampered to date by the lack of success in efficiently generating stable loss-of-function phenotypes. The ability to determine the impact caused in a living organism by lack of expression of a particular gene product promises to greatly facilitate understanding of mammalian gene regulation and gene function. This ability to dissect mammalian genetic pathways will ultimately enable the identification of targets for therapeutic interventions aimed at compensating for genetic deficiencies.

Viral vectors capable of transferring genetic material into mammalian cells have the potential to provide a wide range of experimental and therapeutic uses. Lentiviruses are a subgroup of retroviruses capable of infecting non-dividing cells. The human immunodeficiency viruses, HIV-1 and HIV-2, are members of the lentivirus subclass of retroviruses. Lentiviral vector systems based on the human immunodeficiency virus (HIV) can transduce heterologous nucleic acid sequences into mammalian cells and have been successfully used to introduce transgenes into a variety of human cell types, including primary macrophages and terminally differentiated neurons. Given this potential, lentiviral vectors, including HIV-derived vectors, hold great promise for both investigative and therapeutic applications of mammalian genetics.

Normally, when a gene is turned on, or expressed, a series of events is set in motion, which results in the production of a protein. RNA interference disrupts gene expression by targeting an intermediate molecule called mRNA for degradation. RNA interference (RNAi) is a phenomenon in which double-stranded RNA (dsRNA) specifically suppresses the expression of a gene bearing its complementary sequence. Small interfering RNA (siRNA) can be used to induce RNAi in mammalian cells. RNA interference appears to have evolved as a cellular defense mechanism to suppress viral infection and transposon jumping. In vivo the dsRNA intermediates, by the action of an endogenous ribonuclease, are reduced to siRNAs that are the actual mediators of the RNAi effect. During this process, a hairpin-shaped siRNA molecule binds to mRNA, causing its removal. As a result, little or no protein is produced and thus gene expression is silenced.

RNAi has been used extensively to characterize genes in C. elegans and D. melanogaster. RNAi is typically induced in these organisms by the introduction of long dsRNA produced by in vitro transcription. Attempts to use this approach in mammalian cells, however, have failed because introducing long dsRNAs into mammalian cells elicits a very strong anti-viral response. This anti-viral response causes a global change in gene expression, obscuring any gene-specific silencing that may otherwise be occurring. However, siRNAs do not stimulate the anti-viral response, and can effectively target specific RNA for gene silencing.

The ability to achieve reliable and efficient delivery of siRNA to cellular systems would render siRNA a powerful functional genomics tool by providing the ability to inhibit expression of target genes to see what effect their absence has on the cell or organism. Furthermore, the ability to selectively inhibit target gene expression has important therapeutic implications and could be useful to prevent the production of proteins that are harmful to the body. For instance, the potential to knock out gene expression holds tremendous potential for treatment of dominant inherited diseases where one mutated copy of a gene dominates the normal gene and causes the genetic disorder in the offspring. In these diseases, siRNA carries the potential to specifically degrade mRNA that corresponds to mutant genes involved in disease, shutting off the harmful effects of the proteins they encode.

Compared to conventional methods of inhibiting gene expression siRNA has significant potential for therapeutic success. In this regard, siRNA is significantly more stable than single-stranded antisense molecules, making cellular delivery easier. The stability of siRNA allows for higher efficiency at getting to and eliminating gene targets than antisense oligonucleotides. Significantly, if efficiently delivered to a cell of interest, siRNA can effect gene-silencing of a target gene through mRNA degradation. This represents an advantage over its natural precursor, dsRNA, which causes a nonspecific response. In addition, siRNA is more stable than single-stranded antisense molecules, making cellular delivery easier.

Thus, there exists a need for creating methods for efficiently and reliably delivering siRNA to cellular systems. The present invention satisfies this need and provides related advantages as well.

### SUMMARY OF THE INVENTION

The invention provides a lentiviral vector capable of inhibiting the expression of at least one target gene. A lentiviral vector of the invention encompasses a first nucleic acid sequence derived from a target gene transcript and a second nucleic acid sequence corresponding to the reverse complement of the first nucleic acid sequence. A lentiviral vector of the invention capable of inhibiting the expression of at least one target gene is useful in therapeutic applications to inactivate disease-associated transcripts and thereby reduce the severity of inherited metabolic, infectious or malignant conditions.

The invention also provides a lentiviral vector production system. The lentiviral vector production system includes (a) a packaging component encompassing nucleic acid sequences encoding lentiviral structural polypeptides required to generate a lentiviral vector, and (b) a transfer vector component encompassing cis-acting nucleic acid sequences necessary for viral transduction and further encompassing a first nucleic acid sequence derived from a target gene transcript and a second nucleic acid sequence corresponding to the reverse complement of said first nucleic acid sequence. The first and second nucleic acid sequences can be expressed from an exogenous viral or cellular promoter that is inserted into the lentiviral vector.

Also provided by the invention are in vivo and in vitro methods of inhibiting the expression of a target gene in a cell by contacting a cell under conditions that permit infection with a lentiviral vector encompassing a first nucleic acid sequence derived from a target gene transcript and a second nucleic acid sequence corresponding to the reverse complement of the first nucleic acid sequence under the control of at least one promoter capable of, for example, mammalian expression. The invention also provides methods for producing a non-human mammal in which the expression of a target gene is inhibited.

The invention, in a further embodiment, provides a method of selecting a compound that potentially reduces or eliminates a condition associated with a decrease in expression of a gene product by administering an agent to a transgenic non-human mammal in which the expression of a target gene is inhibited and determining whether the compound reduces or eliminates said condition.

### BRIEF DESCRIPTION OF THE DRAWINGS

FIG. 1 shows a diagram of a lentiviral vector useful in the invention.

FIG. 2 shows infection of 293T cells with lentivirus expressing GFP from CMV or CAG promoter alone or co-transfected with lentivirus expressing siGFP. FIG. 2A shows target gene inhibition upon infection with 100 ηg of p24 of GFP virus alone or with 100 ηg p24 siGFP-containing virus; Panel 2B shows target gene inhibition upon infection with 10 ηg of p24 of GFP virus alone or with 100 ηg p24 siGFP-containing virus; and panel 2C shows FACS analysis of infected cells with the GFP transducted cells shown by the dark curve and the cells transducted with both GFP and siGFP-containing vectors shown by the light overlaid curve.

FIG. 3 shows inhibition of GFP in 293T cells infected with 100 ηg, 25 ηg and 6.25 ηg of p24 virus and uninfected cells visualized by fluorescence microscopy at ×5 magnification (panel A), ×32 magnification (panel C) and by light microscopy at ×5 magnification (panel B).

FIG. 4 shows inhibition of GFP in primary mouse keratinocytes transducted with lentivirus expressing GFP from either CMV or a CAG promoter, either alone or by co-transfection with siGFP and visualized by fluorescence microscopy at ×5 magnification, ×32 magnification.

FIG. 5 shows target gene inhibition of the GFP gene by siGFP in rat brain primary hypothalamus cells transducted with lentivirus expressing GFP from either CMV or a CAG promoter, either alone or by co-transfection with siGFP and visualized by fluorescence microscopy at ×5 magnification and ×32 magnification.

FIG. 6 shows (a) siGFP treated eggs visualized by fluorescence microscopy at ×32 magnification, and (b) untreated eggs visualized by fluorescence microscopy at ×32 magnification.

FIG. 7 shows (panel A) an siGFP affected pup compared to an unaffected pup, and (panel B) an affected pup showing a patchy chimera pattern of GFP expression.

FIG. 8 shows 293 T cells transducted with a lentiviral vector carrying GFP-CMV either with (L-CMV-GFP-hH1 sip53) or without (L-CMV-GFP) an hH1sip53 cassette insert.

FIG. 9 shows a schematic of a wild type loxP site and a mutant loxP site that contains two nucleotide changes and corresponds to the mU6 TATA box sequence. Also shown are sequences corresponding to the human and murine H1 promoters and a X. laevis promoter sequence.

FIG. 10 shows a loxP-TATA stuffer construct consisting of a mU6 promoter, a loxP TATA flanked stuffer nucleic acid sequence and an siRNA hairpin and a loxP-TATA siGFP construct consisting of a mU6 promoter, one loxpTATA site and an siRNA hairpin. Delivery of CRE recombinase results in excision of the stuffer nucleic acid sequence and converts the loxP-TATA stuffer construct to the loxP-TATA siGFP construct. The lower panel shows fluorescence micrographs demonstrating GFP target gene inhibition.

FIG. 11 shows quantitation by FACS analysis of GFP levels in the presence of the different constructs, in particular, GFP target inhibition with the LoxP-TATA siGFP (IpT siGFP) construct compared to lack of target inhibition by the LoxP-TATA stuffer (S-siGFP) construct.

FIG. 12 shows the effects of transducing 293T cells, which stably express GFP, with two lentiviral vectors, L25 and L27. The L25 vector carries a silencing cassette against GFP in the OFF configuration, in particular, a mU6 promoter, a loxP flanked stuffer and a siRNA against GFP. The L27 vector expresses CRE recombinase. GFP positive cells were transduced with decreasing amounts of L25 and a fixed amount of L27.

FIG. 13 shows FACS quantitation of GFP levels in 293T cells, which stably express GFP, eight days after infection with two lentiviral vectors, L25 and L27. The L25 vector carries a silencing cassette against GFP in the OFF configuration, in particular, a mU6 promoter, a loxP flanked stuffer and a siRNA against GFP. The L27 vector expresses CRE recombinase. GFP positive cells were transduced with decreasing amounts of L25 and a fixed amount of L27. GFP levels are inversely correlated to the amount of L25.

### DETAILED DESCRIPTION OF THE INVENTION

This invention is directed to a lentiviral vector capable of inhibiting the expression of at least one target gene. A lentiviral vector of the invention encompasses a first nucleic acid sequence derived from a target gene transcript and a second nucleic acid sequence corresponding to the reverse complement of the first nucleic acid sequence. Once delivered to a cell, expression of the first nucleic acid sequence derived from the target gene transcript and the second nucleic acid sequence corresponding to the reverse complement of the first nucleic acid sequence results in formation of a double-stranded siRNA that inhibits the expression of the target gene. Introduction of a lentiviral vector of the invention into a cell is useful to inhibit the expression of a target gene through the process of RNA interference (RNAi) and allows for inhibition of gene expression in a sequence dependent fashion. As shown herein, the lentiviral vectors provided by the present invention can efficiently deliver nucleic acid sequences to mammalian cells and allow for stable expression of siRNA.

In certain embodiments, a lentiviral vector of the invention can encompass nucleic acid sequences sufficient to form more than one double-stranded siRNA that inhibit expression of distinct target genes. In this embodiment, simultaneous inhibition of distinct target genes can be accomplished with a single lentiviral vector of the invention. The number of different siRNA transcripts that can be expressed simultaneously is limited only by the packaging capacity of the lentiviral vector and adjacent promoters, including any of the promoters described below, can be selected to eliminate or minimize interference and allow for efficient simultaneous inhibition of multiple target genes. The inhibition of multiple siRNA transcripts of adjacent promoters, for example, 2 or more, 3 or more, 4 or more, 5 or more, 6 or more, 7 or more, 8 or more, 9 or more, or 10 or more adjacent promoters allows the user to generate a desire phenotype that develops only when several genes are targeted simultaneously and enables manipulation and elucidation of complex genetic systems.

In addition, a single cell can be co-transducted with more than one lentiviral vector of the invention. Therefore, more than one target gene can be inhibited in a cell, either by transduction with a single lentiviral vector or by co-transduction with more than one lentiviral vector of the invention.

Protein synthesis involves two steps, transcription and translation. First, during transcription, genes are copied from double-stranded deoxyribonucleic acid (DNA) molecules into mobile, single-stranded ribonucleic acid (RNA) molecules called messenger RNA (mRNA). Subsequently, during translation, mRNA is converted into functional proteins. Since there are two steps to making a protein, there are two principal approaches to preventing protein production. By delivering to a cell double-stranded RNA duplexes with very short 3′ overhangs that correspond to siRNA molecules it is possible to trigger RNA interference and block protein synthesis at the translation step.

RNAi is a natural phenomenon believed to occur in the nematode Caenorhabditis elegans, in the fruit fly Drosophila melanogaster, and in some plant species. It most likely serves to protect organisms from viruses, and suppress the activity of transposons, segments of DNA that can move from one location to another, sometimes causing production of an abnormal gene product. An intermediate in the RNAi process, siRNA can be effective in degrading mRNA in mammalian cells and, therefore, carries the potential to specifically degrade mRNA that corresponds to a target gene and thereby inhibit its expression. The strand of the siRNA that is identical in sequence to a region on a target gene transcript is often referred to as the sense strand, while the other strand, which is complementary, is frequently termed the antisense strand.

In RNA interference as it occurs naturally, during the initiation step, input dsRNA is digested into 21-23 nucleotide small interfering RNAs (siRNAs), which have also been called “guide RNAs” as described in Hammond et al. Nature Rev Gen 2: 110-119 (2001); Sharp, Genes Dev 15: 485-490 (2001); and Hutvagner and Zamore, Curr Opin Genetics & Development 12:225-232(2002), which are incorporated herein by reference in their entirety. The siRNAs are produced when an enzyme belonging to the RNase III family of dsRNA-specific ribonucleases progressively cleaves dsRNA, which can be introduced directly or via a transgene or vector. Successive cleavage events degrade the RNA to 19-21 base pair duplexes (siRNAs), each with 2-nucleotide 3′ overhangs as described by Hutvagner and Zamore, Curr. Opin. Genetics & Development 12:225-232 (2002); Bernstein et al., Nature 409:363-366 (2001), which are incorporated herein by reference in their entirety. In the effector step, the siRNA duplexes bind to a nuclease complex to form what is known as the RNA-induced silencing complex, or RISC. The active RISC then targets the homologous transcript by base pairing interactions and cleaves the mRNA approximately 12 nucleotides from the 3′ terminus of the siRNA (Nykanen et al., Cell 107:309-321 (2001), which is incorporated herein by reference in its entirety).

In most mammalian cells dsRNA provokes a non-specific cytotoxic response. In contrast, the introduction of siRNAs, as provided by the present invention, appears to suppress gene expression without producing a non-specific cytotoxic response because the small size of the siRNAs, as compared to dsDNA, prevents activation of the dsRNA-inducible interferon system in mammalian cells and avoids the non-specific phenotypes that can be observed by introducing larger dsRNA.

As used herein, the term “target gene transcript” refers to a single-stranded RNA copy that has substantially the same nucleic acid sequence as a portion of coding or sense strand sequence of a target gene, except for possessing Uracil instead of Thymine. A target gene transcript has substantially the sequence that would result during mRNA synthesis from the template or antisense strand that corresponds to a portion of the target gene. A target gene transcript can have, for example, between 50 and 100 contiguous nucleotides, between 25 and 50 contiguous nucleotides, between 14 and 26 contiguous nucleotides that correspond to the target DNA, between 15 and 25, between 16 and 24, between 17 and 23, between 18 and 22, between 19 and 21 contiguous nucleotides, up to the full length transcript, as long as the resulting double-stranded target gene transcript is capable of specific target gene inhibition. In this regard, the target gene transcript can be of any length as long dsRNA-dependent protein kinase (PKR) is not induced upon formation of the double-stranded target gene transcript. A major component of the mammalian nonspecific response to dsRNA is mediated by the dsRNA-dependent protein kinase, PKR, which phosphorylates and inactivates the translation factor eIF2a, leading to a generalized suppression of protein synthesis and cell death via both nonapoptotic and apoptotic pathway. PKR may be one of several kinases in mammalian cells that can mediate this response.

As used herein, the term “reverse complement” when used in reference to a first nucleic acid sequence derived from a target gene transcript refers to the complementary sequence of the first nucleic acid sequence as dictated by base-pairing, but in reverse orientation so as to result in complementarity upon fold-over into the hairpin structure. The term encompasses partial complementarity where only some of the bases are matched according to base pairing rules as well as total complementarity between the two nucleic acid sequences. The degree of complementarity between the first and second nucleic acid sequences can have significant effects on the efficiency and strength of inhibition of the target gene by the resulting double-stranded target gene transcript.

In contrast, the complement of a first nucleic acid sequence derived from a target gene transcript refers is the complementary sequence as dictated by base-pairing. A lentiviral vector capable of inhibiting the expression of a target gene can encompass a first nucleic acid sequence derived from a target gene transcript and a second nucleic acid sequence corresponding to the complement of the first nucleic acid sequence in those embodiments of the invention where the nucleic acid sequences are not expressed from a single transcriptional unit and, consequently, do not fold over into a hairpin structure.

The terms “double-stranded target gene transcript” and “double-stranded siRNA transcript” can be used interchangeably and both refer to a short duplex consisting of the first and second nucleic acid sequences corresponding to the target gene transcript and its reverse complement, respectively. Where driven off separate promoters rather than a single promoter, the terms refer to a short duplex consisting of the first and second nucleic acid sequences corresponding to the target gene transcript and its complement, respectively. A double-stranded target gene transcript corresponds to an siRNA of the target gene. The term encompasses both partially or completely double-stranded transcripts. Generally, a siRNA encompasses to fragments of at least 18, at least 19, at least 20, at least 21, at least 22, at least 23, at least 24, at least 25, at least 30, at least 35, at least 40, at least 45, at least 50 or more nucleotides per strand, with characteristic 3′ overhangs of at least 1, at least 2, at least 3, or at least 4 nucleotides. As set forth above, a double-stranded target gene transcript can be of any length desired by the user as long as the ability to inhibit target gene expression is preserved.

As used herein, the term “replication-defective” when used in reference to a lentiviral vector means that the vector is incapable of spreading after the initial infection. A lentiviral vector can be modified by replacement, alteration or omission of coding or regulatory regions that render the lentivirus incapable of making the proteins required for replication.

As used herein, the term “transfection” refers to the introduction of a nucleic acid sequence into a eukaryotic cell. Transfection can be accomplished by a variety of means known to the art including but not limited to calcium phosphate-DNA co-precipitation, DEAE-dextran-mediated transfection, polybrene-mediated transfection, electroporation, microinjection, liposome fusion, lipofection, protoplast fusion, and biolistics.

As used herein, the term “transduction” refers to the delivery of a nucleic acid sequence using a lentiviral vector by means of infection rather than by transfection.

As used herein, the term “vector” refers to a nucleic acid molecule capable of transferring another nucleic acid sequence to which it has been linked. The term is intended to include any vehicle for delivery of a nucleic acid, for example, a virus, plasmid, cosmid or transposon.

The term “component” as used in reference to a lentiviral production system of the invention, is meant to refer to one or more physically separate constructs, which can be part of a vector production system of the invention. For example, the nucleic acid sequences encoding polypeptides having virus packaging functions necessary for generation of a lentiviral vector of the invention can be divided onto separate expression plasmids that are independently transfected into the packaging cells.

Retroviridae encompass a large family of RNA viruses that is, in part, characterized by its replicative strategy, which includes as essential steps reverse transcription of the virion RNA into linear double-stranded DNA and the subsequent integration of this DNA into the genome of the cell. Retroviruses are defined by common taxonomic denominators that include structure, composition, and replicative properties. Retroviruses further encompass simple and complex retroviruses, which can be distinguished by the organization of their genomes.

All retroviruses contain three major coding domains with information for virion proteins: gag, which directs the synthesis of internal virion proteins that form the matrix, the capsid, and the nucleoprotein structures; pol, which contains the information for the reverse transcriptase and integrase enzymes; and env, from which are derived the surface and transmembrane components of the viral envelope protein. An additional, smaller, coding domain present in all retroviruses is pro, which encodes the virion protease. The term encompasses viruses that can be subdivided into seven groups defined by evolutionary relatedness, each with the taxonomic rank of genus. Five of these genuses, Avian sarcoma and leukosis viral group, Mammalian B-type viral group, Murine leukemia-related viral group, Human T-cell leukemia-bovine leukemia viral group, and D-type viral group, represent retroviruses with oncogenic potential, formerly referred to as oncoviruses. The other two genuses encompassed by the term are the lentiviruses and the spumaviruses, which are complex retroviruses. A retroviral vector of the invention can have at least one of the functional characteristics associated with the family.

The present invention, while applicable to any retroviral vector, is specifically directed to lentiviral vectors, also referred to as a lentivectors. As used herein the term “lentivirus” refers to a genus of retroviruses, that is distinguishable from other members of the family based on a variety of characteristics, for example, virion morphology, host range, genome organization and pathological effects. For example, the morphology of a lentiviral virion is distinct from other retroviruses based on its cone-shaped core.

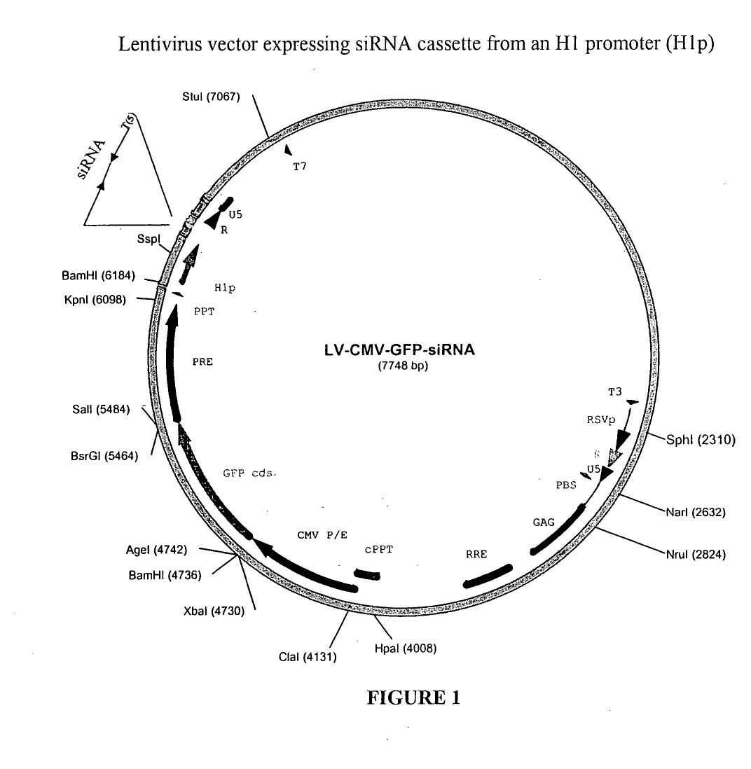

As used herein, the term “lentiviral vector” refers to a modified lentivirus, for example, a HIV-1, that is used to introduce a nucleic acid sequence into a cell. A lentiviral vector retains at least one of the functional characteristics of the virus from which it is derived and can further be modified to exhibit additional functional characteristics. Modifications can include, for example, expansion of host cell range; modulation of the ability to infect other cells; and incorporation of heterologous polypeptides. FIG. 1 shows a diagram of a lentiviral vector useful in the invention.

As used herein, the term “lentiviral polypeptide” encompasses the multiple proteins encoded by, for example, viral gag, pol and env genes which are typically expressed as a single precursor. For example, HIV gag encodes, among other proteins, p 17, p24, p9 and p6; HIV pol encodes, among other proteins, protease (PR), reverse transcriptase (RT) and integrase (IN); and HIV env encodes, among other proteins, Vpu, gp120 and gp41. The term is meant to encompass all or any portion of the reference lentiviral polypeptide as long as at least one functional characteristic of the polypeptide is retained.

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants. Mammalian cell lines useful in the invention include established mammalian cell lines, such as 293T, COS, CHO, HeLa, NIH3T3 and PC12 cells as well as cell lines derived and established while practicing the invention.

As described herein, a lentiviral vector of the invention contains a first nucleic acid sequence derived from a target gene transcript and the reverse complement of the first nucleic acid sequence. In a particular embodiment, the invention provides a lentiviral vector, wherein the first and second nucleic acid sequences are each between 19 and 22 nucleotides in length. As set forth above, the nucleic acid sequences can be of any length desired by the user as long as the resulting double-stranded target gene transcript is capable of specific target gene inhibition. For example, the target gene transcript can be of any length as long double-stranded RNA-dependent protein kinase (PKR) is not induced upon formation of the double-stranded target gene transcript. A major component of the mammalian nonspecific response to double-stranded RNA is mediated by the ds RNA-dependent protein kinase, PKR, which phosphorylates and inactivates the translation factor eIF2a, leading to a generalized suppression of protein synthesis and cell death via both nonapoptotic and apoptotic pathway.

Upon introduction of the lentiviral vector into a cell, the first and second nucleic acid sequences form one or more RNA duplexes referred to as siRNAs that correspond to one or more target genes and inhibit the expression of the target gene(s). Thus, a lentiviral vector of the invention is useful for introduction of a nucleic acid molecule corresponding to a short double stranded RNA (dsRNA), that is homologous in sequence to and capable of inhibiting expression of at least one target gene.

Among its embodiments, the invention provides lentiviral vectors and lentiviral production systems. Lentiviruses are diploid positive-strand RNA viruses of the family Retroviridae that replicate through an integrated DNA intermediate. In particular, upon infection by the RNA virus, the lentiviral genome is reverse-transcribed into DNA by a virally encoded reverse transcriptase that is carried as a protein in each lentivirus. The viral DNA is then integrated pseudo-randomly into the host cell genome of the infecting cell, forming a provirus that is inherited by daughter cells. Known lentiviruses can be readily obtained from depositories or collections such as the American Type Culture Collection (“ATCC”; 10801 University Blvd., Manassas, Va. 20110-2209), or isolated from known sources using commonly available techniques. While described with reference to lentiviral vectors, the present invention encompasses any known retrovirus that can be readily utilized given the disclosure provided herein and standard recombinant techniques as described in Sambrook et al., Molecular Cloning: A Laboratory Manual, 3rd ed., Cold Spring Harbor Press, Plainview, N.Y. (2001) and in Ausubel et al., Current Protocols in Molecular Biology (Supplement 47), John Wiley & Sons, New York (1999), both of which are incorporated herein by reference in their entirety.

Functional characteristics of a lentivirus include, for example, infecting non-dividing host cells, transducing non-dividing host cells, infecting or transducing host immune cells, containing a lentiviral virion including one or more of the gag structural polypeptides p7, p24 or p17, containing a lentiviral envelope including one or more of the env encoded glycoproteins p41, p 120 or p 160, containing a genome including one or more lentivirus cis-acting sequences functioning in replication, proviral integration or transcription, containing a genome encoding a lentiviral protease, reverse transcriptase or integrase, or containing a genome encoding regulatory activities such as Tat or Rev. A lentiviral vector of the invention will exhibit at least one of the functional characteristics of the genus and can be derived from any appropriate lentivirus, for example, a human immunodeficiency virus-1 (HIV-1), human immunodeficiency virus-2 (HIV-2), simian immunodeficiency virus (SIV), feline immunodeficiency virus (FIV) and equine infectious anemia virus (EIAV).

As described herein, the introduction into a cell of a double stranded target gene transcript corresponding to an siRNA, which is delivered by a lentiviral vector of the invention and which is formed by first a nucleic acid sequence derived from a target gene transcript and a second nucleic acid sequence corresponding to the reverse complement of the first nucleic acid sequence, can inhibit expression of a target gene as a result of mRNA inhibition or degradation.

A target gene can be any gene that is present and expressed in the cell, provided that at least such part of the target gene sequence is known as is sufficient to allow selection of the nucleic acid sequence corresponding to the target gene transcript. Thus, it is not required that the entire sequence of the target gene is known to the user practicing the invention.

The nucleic acid sequence derived from a target gene transcript can be selected based on a variety of considerations. To select the nucleic acid sequence either part of or the entire target gene sequence can be scanned and potential sequence sites can be recorded. Potential sequence sites can then be evaluated by a BLAST analysis against the GENBANK database to disqualify any target sequence with significant homology to other genes. Furthermore, siRNAs can be designed to regions of target mRNA with low secondary structure. If desired, two or more nucleic acid sequences can be selected for preparation of separate lentiviral vectors capable of inhibition of a target gene. This approach allows for comparison of the efficiency of target gene inhibition between the nucleic acid sequences representing the target gene transcript.

Two approaches can be used for expressing a double stranded siRNA transcript. In the first, the nucleic acid sequence constituting the siRNA duplex are transcribed by individual promoters that drive their expression. In the second, the first and second nucleic acid sequences are expressed off a single promoter resulting in a fold-back stem-loop or hairpin structure that is processed into the siRNA.

A lentiviral vector or vector production system of the invention can utilize any promoter desired by the user as appropriate for the expression context. A promoter useful in the present invention can comprise a promoter of eukaryotic or prokaryotic origin that can provide high levels of constitutive expression across a variety of cell types and will be sufficient to direct the transcription of a distally located sequence, which is a sequence linked to the 5′ end of the promoter sequence in a cell. The promoter region can also include control elements for the enhancement or repression of transcription and can be modified as desired by the user and depending on the context. Suitable promoters include, for example, RNA polymerase (pol) III promoters including, but not limited to, the human and murine U6 pol III promoters as well as the human and murine H1 RNA pol III promoters; RNA polymerase (pol) II promoters; cytomegalovirus immediate early promoter (pCMV), the Rous Sarcoma virus long terminal repeat promoter (pRSV), and the SP6, T3, and T7 promoters. In addition, a hybrid promoter also can be prepared that contains elements derived from, for example, both a RNA polymerase (pol) III promoter and an RNA polymerase (pol) II promoter. Modified promoters that contain sequence elements derived from two or more naturally occurring promoter sequences can be combined by the skilled person to effect transcription under a desired set of conditions or in a specific context.

Enhancer sequences upstream from the promoter or terminator sequences downstream of the coding region can be optionally be included in the vectors of the present invention to facilitate expression. Vectors of the present invention can also contain additional nucleic acid sequences, such as a polyadenylation sequence, a localization sequence, or a signal sequence, sufficient to permit a cell to efficiently and effectively process the protein expressed by the nucleic acid of the vector. Such additional sequences can be inserted into the vector such that they are operably linked with the promoter sequence, if transcription is desired, or additionally with the initiation and processing sequence if translation and processing are desired. Alternatively, the inserted sequences can be placed at any position in the vector.

As used herein, an “inducible promoter” refers to a transcriptional control element that can be regulated in response to specific signals. An inducible promoter is transcriptionally active when bound to a transcriptional activator, which in turn is activated under a specific set of conditions, for example, in the presence of a particular combination of chemical signals that affect binding of the transcriptional activator to the inducible promoter and/or affect function of the transcriptional activator itself. Thus, an inducible promoter is a promoter that, either in the absence of an inducer, does not direct expression, or directs low levels of expression, of a nucleic acid sequence to which the inducible promoter is operably linked; or exhibits a low level of expression in the presence of a regulating factor that, when removed, allows high-level expression from the promoter, for example, the tet system. In the presence of an inducer, an inducible promoter directs transcription at an increased level.

The function of a promoter can be further modified, if desired, to include appropriate regulatory elements to provide for the desired level of expression or replication in the host cell. For example, appropriate promoter and enhancer elements can be chosen to provide for constitutive, inducible or cell type-specific expression. Useful constitutive promoter and enhancer elements for expression of a target gene transcript include, for example, RSV, CMV, CAG, SV40 and IgH elements. Other constitutive, inducible and cell type-specific regulatory elements are well known in the art.

A promoter that is particularly useful in the lentiviral vector of the invention is compatible with mammalian genes and, further, can be compatible with expression of genes from a wide variety of species. For example, a promoter useful for practicing the invention can be a promoter of the eukaryotic RNA polymerases pol II and pol III, or a hybrid thereof. The RNA polymerase III promoters have a transcription machinery that is compatible with a wide variety of species, a high basal transcription rate and recognize termination sites with a high level of accuracy. For example, the human and murine U6 RNA polymerase (pol) III and H1 RNA pol III promoters are well characterized and useful for practicing the invention. As exemplified below, because the activities of these two promoters as well as the localization of expressed nucleic acid sequences can vary from cell type to cell type, if desired, U6 and H1 lentiviral vectors of the invention can be prepared and targeted to the desired cells for target gene inhibition. One skilled in the art will be able to select and/or modify the promoter that is most effective for the desired application and cell type so as to optimize target gene inhibition.

Thus, promoters that are useful in the invention include those promoters that are sufficient to render promoter-dependent gene expression controllable for cell-type specificity, cell-stage specificity, or tissue-specificity, and those promoters that are inducible by external signals or agents, for example, metallothionein, MMTV, and pENK promoters. The promoter sequence can be one that does not occur in nature, so long as it functions in a mammalian cell.

In particular embodiments, intracellular transcription of siRNAs can be achieved by cloning the siRNA templates into RNA pol III transcription units, which normally encode the smaller nucleic RNA (snRNA) U6 or the human RNAse P RNA H1. The U6 and H1 promoters are members of the type III class of Pol III promoters. The U6 and H1 are different in size but contain the same conserved sequence elements or protein binding sites. The +1 nucleotide of the U6-like promoters is always guanosine, whereas the +1 for H1 promoters is adenosine. The termination signal for these promoters is defined by 5 thymidines, and the transcript is typically cleaved after the second uridine. Cleavage at this position generates a 3′ UU overhang in the expressed siRNA, which is similar to the 3′ overhangs of synthetic siRNAs. Any sequence of up to 400 nucleotides in length can be transcribed by the polIII promoters, therefore they are ideally suited for the expression of the nucleic acid sequences that are subject of the invention. As described below, lentiviral siRNA-containing vectors of the invention can experience stable, long-term target gene inhibition, whereas cells which are transfected with exogenous synthetic siRNAs typically recover from target gene inhibition within seven days or ten rounds of cell division.

The promoter that drives expression of the siRNA transcript in the target cell can further be useful to restrict expression to a specific time, cell type or tissue. If desired, regulatable transcriptional elements can be incorporated into a lentiviral vector of the invention that can be switched on and off via exogenous stimuli. The regulatable systems can be based on naturally occurring inducible promoters that exhibit tissue specificity or consist of chimeric systems, which contain pro- and eukaryotic elements from different organisms as described in Aga-Mohamad and Lotte, J. Cain. Invest. 105:1177-83 (2000), which is incorporated herein by reference.

The tetracycline-(tet)-regulatable system, which is based on the inhibitory action of the tet repression (tetr) of Escherichia coli on the tet operator sequence (TECO) can be modified for use in mammalian systems and is a useful regulatable element for the lentiviral vectors of the invention (See, Goshen and Badgered, Proc. Natl. Acad. Sci. USA 89: 5547-51 (1992)). Briefly, for use in mammalian cells the tetr is fused to the carboxyl terminus of VP16 (a herpes virus transactivator), and the tECO-repeats are fused to a minimal human CMV promoter. In the presence of tet, the tetRVP16 fusion protein cannot bind to and activate tECO (tet-off system), whereas in the absence of tet, the tetr-VP16 protein can bind to tECO, resulting in increased expression levels of the siRNA transcript. The tet-off system and the siRNA transgene can be contained on a lentiviral vector of the invention. The regulated expression of both the siRNA transcript as well as the regulatory system can be achieved by using an internal ribosomal entry site (IRES), resulting in bi-cistronic expression. Cell toxicity of the tetr-VP16 fusion protein can be overcome by placing tetr-VP16 under the control of the tECO-containing promoter as described by Shockett et al., Proc. Natl. Acad. Sci. USA 92:6522-26 (1996), which is incorporated herein by reference. The reverse tet-regulated system in which the addition of tet induces transactivation (tet-on) also can be used in a lentiviral vector of the invention. See, Lindemann et al., Mol. Med. 3:466-76 (1997), which is incorporated herein by reference.

Thus, for therapeutic applications of the present invention, for example, in somatic gene therapy, the transient controllable expression of a lentiviral vector of the invention can allow for controlled target gene inhibition. In this embodiment, the expression of the siRNA transgene can be induced or suppressed by the simple administration or cessation of administration to an individual, respectively, of an exogenous inducer such as, for example, tetracycline or its derivative doxycycline. In this embodiment, the invention allows for efficient regulation of target gene inhibition, a low background level of inhibition in the off state, fast induction kinetics, and large window of regulation by administering the inducer, for example, tetracycline or a tetracycline analogue to the individual. The level of siRNA expression can be varied depending upon which particular inducer, for example, which tetracycline analogue is used. In addition, the level of siRNA expression can also be modulated by adjusting the dose of the inducer that is administered to the patient to thereby adjust the concentration achieved in the circulation and in the tissues of interest. The inducer can be administered by any route appropriate for delivery of the particular inducing compound and preferred routes of administration can include oral administration, intravenous administration and topical administration.

There are several situations in which it may be desirable to be able to inhibit the target gene at specific levels and/or times in a regulated manner, rather than simply inhibiting the target gene constitutively at a set level. For example, a gene of interest can be inhibited at fixed intervals to provide the most effective level of target gene inhibition at the most effective time. The level of gene product produced in a subject can be monitored by standard methods, for example, direct monitoring using an immunological assay such as ELISA or RIA or indirectly by monitoring of a laboratory parameter dependent upon the function of the gene product of interest, for example, blood glucose levels. The ability to effect target gene inhibition at discrete time intervals in a subject allows for focused treatment of conditions only at times when treatment is necessary, for example, during the acute phase or during a particular stage of development.

It is further contemplated that an inducible lentiviral vector of the invention can be prepared by incorporating a recombination system, for example, the Cre/lox system of bacteriophage P1, the FLP/FRT system of the yeast 2 uM plasmid, the R/RS system of the yeast plasmid pSR1, or the modified Gin/gix system of bacteriophage Mu. In a particular embodiment exemplified herein, an inducible lentiviral vector of the invention is prepared that incorporates the Cre/loxP recombination system. Briefly, Cre is a 38 kDa recombinase protein from bacteriophage P1 which mediates intramolecular (excisive or inversional) and intermolecular (integrative) site specific recombination between loxP sites as described by Sauer, Methods of Enzymology, 225: 890-900(1993), which is incorporated herein by reference. A loxP site (locus of X-ing over) consists of two 13 bp inverted repeats separated by an 8 bp asymmetric spacer region. One molecule of Cre binds per inverted repeat or two Cre molecules line up at one loxP site. The recombination occurs in the 8 base pair asymmetric spacer region, which also is responsible for the directionality of the site. Two loxP sequences in opposite orientation to each other invert the intervening piece of DNA, two sites in direct orientation dictate excision of the intervening DNA between the sites leaving one loxP site behind.

The ability to excise a piece of nucleic acid sequence at a particular time can be exploited by flanking a nucleic acid sequence with a pair of lox P sites and introduce the cre enzyme when excision is desired. If desired, a Cre transgene can be put under control of an inducible and/or tissue specific promoter to allow excision of a nucleic acid sequence in selected cells and at selected times. As described herein, an inducible lentiviral vector of the invention can include a nucleic acid sequence that serves as a stuffer fragment between the promoter and the siRNA hairpin. The stuffer fragment can be flanked by loxP sites, so that a CRE mediated recombination event leads to excision of the stuffer nucleic acid sequence and juxtaposition of the siRNA hairpin and the promoter, resulting in target gene inhibition. An inducible lentiviral vector of the invention can be used for any application, for example, somatic gene therapy where the transient controllable expression of a lentiviral vector of the invention is desirable. In addition, an inducible lentiviral vector of the invention can be used to dissect complex biological problems in vivo, as it allows for inhibition target genes in a tissue specific manner by putting CRE under the control of a tissue specific promoter. In this embodiment of the invention, a lentiviral vector of the invention can be utilized for focused target gene inhibition in specific regions of a tissue.

Thus, a lentiviral vector of the invention can further encompass nucleic acid sequences sufficient for induction by a site-specific recombinase, for example, cre recombinase. In this embodiment induction of the promoter that drives siRNA expression is initiated by contacting the lentiviral vector with a recombinase that mediates a recombination event that involves excision of a stuffer nucleic acid sequence and results in juxtaposition of the promoter and the corresponding first and second nucleic acid sequences driven by the promoter so as to allow transcription and formation of a double-stranded siRNA transcript capable of inhibiting the expression of a target gene.

As used herein in reference to an inducible lentiviral vector of the invention, the terms “stuffer nucleic acid sequence” and “stuffer fragment” refer to a nucleic acid sequence that is inserted into or proximal to a promoter sequence driving siRNA nucleic acid sequence expression and that further contains a transcription stop signal specific to the promoter. The presence of the stuffer fragment thus prevents transcription of the siRNA nucleic acid sequences off their corresponding promoter and keeps the promoter-siRNA nucleic acid construct in a non-induced, inactive state. Conversely, upon addition of a recombinase enzyme site specific excision of the stuffer fragment containing the promoter specific transcription stop signal results in juxtaposition of the promoter and its corresponding siRNA nucleic acid sequences, resulting in transcription of the siRNA nucleic acid sequences and expression of the double-stranded trasncript capable of target gene inhibition.

A stuffer fragment can be any nucleic acid sequence and preferably is a a relatively inert sequence that is not prone to conformational changes. For example, a stuffer sequence can be a segment of the lacZ gene or any other desired nucleic acid segment provided the transcription stop signal that is specific to the promoter driving the siRNA nucleic acid transcription is encompassed and functional in preventing transcription. If desired by the user, the stuffer fragment can contain additional features, for example, a selectable marker that allows for easy detection and determination of the transcriptional state as induced versus non-induced.

The size of a stuffer fragment can be 500 base pairs or more, 600 base pairs or more, 700 base pairs or more, 800 base pairs or more, 1000 base pairs or more, 1200 base pairs or more, 1400 base pairs or more, as long as there is no impairment of its ability to prevent transcription through presence of the promoter specific transcription stop signal or being excised in an enzyme mediated recombination event. An exmple of a stuffer fragment is a 1 kilo base segment of the lacZ gene that contains a sequcence consisting of five adjacent Thymines corresponding to a mU6 promoter specific transcription stop signal.

Additional chimeric-regulated systems useful in the invention are known in the art and include, for example, the progesterone system is based on a mutated human progesterone receptor; the insect ecdysone-responsive system; the Bombyx-derived ecdysone-responsive system (BmEcR); and the rapamycin-regulated transcriptional system. These and other regulatable systems are known in the art and have been described, for example, by Wang et al., Proc. Natl. Acad. Sci. USA 91:8180-84 (1994); No et al., Proc. Natl. Acad. Sci. USA 93:3346-51 (1996); Suhr et al., Proc. Natl. Acad. Sci. USA 95:7999-8004 (1998); Pollock et al., Proc. Natl. Acad. Sci. USA 97:13221-26 (2000), all of which are incorporated herein by references.

In one embodiment of the invention, the transcription of the first nucleic acid sequence derived from the target gene transcript and of its reverse complement are driven by a single promoter capable of mammalian expression. In this embodiment, the nucleic acid sequences derived from the target gene transcript and its reverse complement are separated by a short spacer sequence. The spacer sequence can be of any length desired by the user, and can have for example, 2 or more, 3 or more, 4 or more, 5 or more, 6 or more, 7 or more, 8 or more 9 or more, 10 or more, 15 or more, 20 or more, 25 or more, 30 or more, 40 or more nucleotides. If desired, the spacer sequence can further modified to include regulatory elements useful for lentiviral expression. The resulting transcript folds back on itself to form a double-stranded transcript stem-loop, also referred to as hairpin, structure upon complementary base pairing of the nucleic acid sequence derived from the target gene transcript and its reverse complement.

In most applications, it is desired that the lentiviral vector does not continue to spread after the initial infection. As described in more detail below, methods for constructing a non-replicating lentiviral vector are well known in the art and generally include replacement of most or all of the coding regions of a lentivirus with the gene(s) or sequence elements to be transferred, so that the vector by itself is incapable of making proteins required for additional rounds of replication. As described herein, viral proteins needed for the initial infection can be provided in trans by a lentiviral packaging cell obviating the need for lentiviral protein synthesis in recipient cells for proviral integration.

Lentiviral vectors have demonstrated efficient and long-lasting transfer of nucleic acid sequences into a variety of mammalian cells, including both dividing and non-dividing cells such as nerve, liver, muscle and bone marrow stem cells. As used herein the term “non-dividing” when used in reference to a cell means that the cell does not undergo mitosis. Naturally occurring non-dividing cells include, for example, neurons, hepatocytes, muscle fibers and non-proliferating hematopoeitic cells. The term also encompasses cells that where actively dividing and in which cell-division was blocked by artificial means and terminally differentiated cells. Thus, the term encompasses cells that, either naturally or as a result of manipulation, do not undergo mitosis. The term further encompasses cells that do not undergo mitosis without regard to the means used to block cell division or the point in the cell cycle at which the arrest occurs.

Transduction of cells with a lentiviral vector of the invention can occur either in vitro or in vivo. As used herein, the term “in vivo” means an environment within a living organism or living cell. Such a living organism can be, for example, a multi-cellular organism such as a rodent, mammal, primate or human or another animal such as an insect, worm, frog or fish, or a uni-cellular organism such as a single-celled protozoan, bacterium or yeast. The transducted cell can be in an in utero animal, or in an ex utero animal. Both living cells derived from an organism and used directly (primary cells) as well as cells grown for multiple generations or indefinitely in culture are encompassed within the term “in vivo” as used herein. As an example, an oocyte removed from an organism such as a mouse or a frog and used directly or grown in a tissue culture dish constitutes an in vivo environment.

In vivo applications of the invention include applications in which a target gene transcript is expressed, for example, in a mammalian, primate, human, murine, porcine, bovine, yeast or bacterial cell, for example, a non-human mammalian fertilized oocyte or non-human mammalian embryonic stem cell. In vivo applications therefore include those applications in which a target gene transcript is expressed, for example, such as an established mammalian, human, murine, avian, yeast or cell line and including a Chinese hamster ovary (CHO) cell line, human embryonic kidney 293T cell line, primary skin keratinocyte cell line, primary hypothalamus cell line.

It is understood that in vivo applications can be performed with cells expressing endogenous or exogenous target gene. For example, as exemplified herein, target gene inhibition can be assayed by providing the target gene on a separate plasmid that is co-transfected with the lentiviral vector that contains the first nucleic acid sequence derived from the target gene transcript and the second nucleic acid sequence corresponding to the reverse complement of the first nucleic acid sequence.

In vitro applications also are useful in the methods of the invention. As used herein, the term “in vitro” means an environment outside of a living organism or cell. Applications performed, for example, in a microfuge tube, or a 96, 384 or 1536 well plate, or another assay format with purified or partially purified proteins or cellular extracts outside of a living organism are in vitro applications. Thus, applications performed using whole-cell or fractionated extracts derived from lysed cells, or performed with reconstituted systems, are encompassed within the term “in vitro” as used herein. Furthermore, applications performed in cells or tissues that have been fixed and are therefore dead, denoted in situ assays, for example, utilizing embryoid body-derived cells, also are encompassed within the term “in vitro” as used herein. In view of the above, it is understood that in vitro applications can utilize isolated polypeptides or whole or fractionated cell-free extracts derived, without limitation, from primary cells, transformed cells, cell lines, recombinant cells, mammalian cells, yeast cells or bacterial cells.

In a therapeutic embodiment of the present invention a lentiviral vector can be useful for in vivo delivery and expression of a siRNA corresponding to a target gene transcript into both dividing and non-dividing cells. Methods for preparation of therapeutically safe third-generation lentiviral vectors are known in the art and include, for example, using only a fraction of the total genes normally present in the parent virus and ensures that the lentiviral vector is non-replicating. The genes that can be removed are genes associated with viral replication and pathogenesis, and their elimination is particularly important for the vectors derived from HIV. The removal of the viral replication and pathogenesis genes does not decrease the gene transfer efficiency of the lentiviral vector. If desired, the removal of these genes can be accompanied by the addition of a built-in self-inactivating safety feature that potentially eliminates the possibility that the vector could replicate or recombine with infectious virus during vector manufacturing or patient treatment.

In a further embodiment the invention also provides a lentiviral vector production system. The lentiviral vector production system includes (a) a core packaging component of nucleic acid sequences corresponding to lentiviral structural proteins; and (b) a transfer vector component encompassing lentiviral cis-acting nucleic acid sequences and further encompassing a nucleic acid sequence derived from a target gene transcript and a nucleic acid sequence corresponding to the reverse complement of the target gene, wherein the packaging and the transfer vector components are sufficient to produce a lentiviral vector capable of inhibiting the expression of a target gene in a cell. The packaging and transfer vector components of a lentiviral production system of the invention are sufficient to produce a lentiviral vector capable of inhibiting the expression of a target gene in a cell.

Efficient gene transduction and integration requires the presence of cis-acting nucleic acid sequences or elements in the retroviral vector: a promoter and a polyadenylation signal; a packaging signal to direct incorporation of vector RNA into virion; a primer-binding site and polypurine tract for initiation and R region for strand transfer during reverse transcription; and sequences at the termini of the viral LTR for integration. Other than the cis-acting elements, all of the coding regions of a retrovirus can be removed. The lentiviral cis-acting nucleic acid sequences or elements perform the transduction functions of a lentiviral vector production system.

A “packaging component” refers to one or more constructs that contain the lentiviral structural polypeptides sufficient for lentiviral vector production. If desired, a split genome packaging strategy in which two or more packaging constructs, for example, one containing gag and pol and the other carrying env, can be used. In addition, the packaging component can contain other polypeptides that function in trans to facilitate, augment or supplement the efficiency of vector production or the functional characteristics of the lentiviral vector particle. A packaging construct can be designed to express some or all of such trans-acting factors stably or transiently.

A lentiviral vector production system of the invention thus encompasses at least two components, the packaging component and the transfer vector component. A lentiviral production system of the invention is sufficient for generating a lentiviral vector capable of inhibiting expression of a target gene upon transduction into a cell. As described herein, each of the two components of a lentiviral production system can further be divided into separate nucleic acid molecules or constructs. The use of separate constructs in preparing the components of a lentiviral production system of the invention and the absence of overlapping sequences between the constructs minimizes the possibility of recombination during vector production. In addition, because no viral proteins are expressed by the lentiviral vector itself, no immune response is triggered against cells expressing vector in animal models.

If desired, a lentiviral vector production system of the invention can further encompass a third component, termed the envelope pseudotype component. In this embodiment, the lentivirus env gene can be deleted from the packaging component and instead the envelope gene of a different virus can be supplied on a third component. As described further below, a commonly used envelope gene is that encoding the G glycoprotein of the vesicular stomatitis virus (VSV-G), which confers stability to the particle and permits the vector to be concentrated to high titers.

Packaging cell lines for vector poduction can be chosen that continuously produce high-titer vector. A packaging cell line useful for producing a lentiviral vector of the invention further can be one in which the expression of packaging genes and VSV-G, and therefore the production of vector, can be turned on at will as described by Kafri et al., J. Virol. 73(1): 576-84 (1999), which is incorporated herein by reference.

A lentiviral vector production system useful in the invention can incorporate a third-generation, Tat-free packaging system as described by Dull et al., Journal of Virology 72:8463-8471 (1998); Pfeifer et al., Procl. Natl. Acad. Sci. USA 97:12227-12232 (2000), which are incorporated herein by reference in their entirety. In addition to the lentiviral structural genes gag, pol and env, naturally occurring HIV contains two regulatory genes, tat and rev, essential for viral replication in the naturally occurring virus and four accessory genes, vif, vpr, vpu and nef, that are not required for viral growth in vitro but are necessary for in vivo replication and pathogenesis.

Briefly, to generate a lentiviral vector of the invention, all of the viral genome can be removed from the virus and replaced by the siRNA nucleic acid sequences. The essential cis-acting sequences, such as the packaging signal sequences, which are required for encapsidation of the vector RNA, can be included in the vector construct. The viral sequences necessary for reverse transcription of the vector RNA and integration of the proviral DNA, the LTRs, the transfer RNA-primer binding site, and the polypurine tract (PPT) can be incorporated into a lentiviral vector of the invention. If desired, further modifications known in the art and described herein can be introduced into a lentiviral vector production system, for example, to effect an increase in viral titers.

As described herein, in a third-generation lentiviral production system of the invention only a fractional set of these lentiviral genes can be used to produce a lentiviral vector of the invention. For example, an HIV-derived vector production system useful in the invention can consist of at least two or more, three or more, four or more separate transcriptional units, which can be located, for example, on separate nucleic acid constructs. The Tat, which serves as a transactivator of the LTR, can be omitted in this system if part of the upstream LTR in the transfer vector is replaced by constitutively active internal promoter sequences, for example, CMV or CAG.

Furthermore, expression of rev in trans can be sufficient with a plasmid that contains only gag and pol coding sequences from HIV. If desired, the first vector component of a lentiviral vector production system of the invention can contain the lentiviral gag, pol and rev genes on one or more separate nucleic acid molecules, for example, plasmids. If rev is deleted from the transfer component of the vector production system, it is necessary to provide the transfer vector and packaging vector with cis acting sequences that replace Rev/RRE function. Furthermore, the transfer vector component of a vector production system of the invention can incorporate a self-inactivating (SIN) LTR rendering the vector itself self-inactivating due to a deletion in a region at the end of the virus genome called the long-terminal repeat (LTR), which describes unique cis-acting sequences that flank the virus genome and are essential to the virus life cycle. A sequence within the upstream LTR serves as a promoter under which the viral genome is expressed. Briefly, the U3 region of the 3′LTR, which harbors the major transcriptional functions of the lentiviral genome, can be deleted. During the process of reverse transcription, the 3′LTR is copied to the 5′LTR. By deleting non-replicative portions of the 3′LTR, the genomic viral DNA is inserted into the target genome as a promoter-less sequence. Inactivation of the promoter activity of the LTR can serve as an important safety feature of the vectors of the invention since it reduces the possibility of insertional mutagenesis.

As described herein, other modifications to enhance safety and specificity include the use of specific internal promoters that regulate gene expression, either temporally or with tissue or cell specificity as well as the introduction of post-transcriptional regulatory elements that enhance expression of the siRNA transcript including, for example, the Woodchuck hepatitis virus post-transcriptional regulatory element (WPRE) and the Cana PPT flap (capped.), as described, for example, by Zephyr et al., J Viol. 1999. 73(4):2886-92; Zennou et al., Cell 101:173-85 (2000), both of which are incorporated herein by reference.

A pseudotyped lentiviral vector capable of inhibiting the expression of a target gene can be produced by transfecting cells with the lentiviral vector production system of the invention. As described herein, exemplary host cells for transfection with the lentiviral vector production system include, for example, mammalian primary cells; established mammalian cell lines, such as COS, CHO, HeLa, NIH3T3, 293T and PC12 cells; amphibian cells, such as Xenopus embryos and oocytes; and other vertebrate cells. Exemplary host cells also include insect cells (for example, Drosophila), yeast cells (for example, S. cerevisiae, S. pombe, or Pichia pastoris) and prokaryotic cells (for example, E. coli).

Methods for introducing a nucleic acid into a host cell are well known in the art and include, for example, various methods of transfection such as calcium phosphate, DEAE-dextran and lipofection methods, electroporation and microinjection. The methods of isolating, cloning and expressing nucleic acid molecules of the invention referred to herein are routine in the art and are described in detail, for example, in Sambrook et al., Molecular Cloning: A Laboratory Manual, Cold Spring Harbor Laboratory, New York (1992) and in Ausubel et al., Current Protocols in Molecular Biology, John Wiley and Sons, Baltimore, Md. (1998), which are incorporated herein by reference. With particular regard to preparation of the first and second nucleic acid sequences corresponding to the target gene transcript, it is understood by those skilled in the art that sequence verification of siRNA templates after cloning is useful, since even a single nucleotide mismatch between the target RNA and the siRNA antisense strand component of the double stranded target transcript can reduce or prevent inhibition.

Fluorescently labeled siRNA can be used to analyze siRNA stability and transfection efficiency. Labeled siRNA is also useful for study of siRNA subcellular localization and, if desired, for applications in which double labeling with a labeled antibody is desired in order to track cells that receive target gene siRNA during transfection or transduction and to correlate transfection or transduction with down-regulation of the target gene. Furthermore, reporter or selectable marker nucleic acid sequences sufficient to permit the recognition or selection of the vector in normal cells are useful components of the lentiviral vectors and vector production systems of the invention. The reporter nucleic sequences can encode an enzyme or other protein that is normally absent from mammalian cells, and whose presence can definitively establish the presence of the vector in such a cell.

A reporter gene useful in the invention encodes for a protein whose activity can be detected with high sensitivity above any endogenous activity and that displays a wide dynamic range of response. It is understood that choosing the appropriate reporter gene depends on the organism and cell type, type of information sought, and preferred detection method. A reporter can be detected via a broad range of assays, including calorimetric, fluorescent, bioluminescent, chemiluminescent, ELISA, and/or in situ staining.

One skilled in the art will be able to select an appropriate reporter or selectable marker based on the cell type, desired sensitivity of detection and method of detection, for example, the genes encoding the green fluorescent protein (GFP), luciferase, beta-galactosidase enzyme (β-gal) and the chloramphenicol acetyl transferase (CAT). In a variety of embodiments of the invention described herein, the GFP gene can be useful marker because it fluoresces green upon irradiation and is an useful in vivo marker of target gene transcript expression because it requires no substrates or co-factors to fluoresce and retains its activity in the presence of heat, denaturants, detergents, and most proteases.

In another embodiment, the invention provides a method of inhibiting the expression of a target gene in a cell by contacting a cell under conditions that permit infection with a lentiviral vector with a nucleic acid sequence derived from a target gene transcript and a nucleic acid sequence corresponding to the reverse complement of the target gene transcript under the control of at least one promoter capable of mammalian expression. Upon transcription, the nucleic acid sequences form a double-stranded target gene transcript that inhibits target gene expression.

It is contemplated that for all embodiments described herein a cell can be co-transducted with more than one lentiviral vector. Similarly, it is contemplated that a single lentiviral vector can encompass nucleic acid sequences derived from more than one target gene. In both approaches the nucleic acid sequences form more than one distinct double-stranded target gene transcript upon transcription and each inhibit the expression of a distinct target gene. It is understood that, in any of the embodiments encompassed by the present invention, more than one target gene can be inhibited either by a single lentiviral vector of the invention or by co-transduction with more than one lentiviral vector.

In a related embodiment, the lentiviral vectors of the invention also can be used in high-throughput in vitro screens for loss-of-function phenotypes. If desired, a population of cells can be prepared that are transducted with a library of lentiviral vectors encompassing different siRNAs. Upon transduction, the cells can be screened for a particular phenotype of interest. In this embodiment, the siRNA can be used to identify genes that, upon inhibition, elicit a particular phenotype indicating their involvement in a process/condition of interest. It is understood that in this embodiment the target gene can be either randomly selected or can be chosen semi-randomly, for example, based on a microarray analysis of the cells chosen for transduction. A cell type of interest can be selected such as, for example, embryonic stem cells, pancreatic cells, cancer cells, and a library of lentiviral vectors can be prepared that encompass nucleic acid sequences selected based on a microarray analysis performed on the particlar cell type. One skilled in the art will be able to select a particular cell type that is known to express the particular genes of interest to be studied, for example, pancreatic cells to screen for genes involved in diabetes.

In further embodiment, the invention provides a method of producing a non-human mammal in which the expression of a target gene is inhibited by (a) infecting a pre-implantation mammalian embryo with a lentiviral vector of described herein, (b) transferring the infected pre-implantation embryo into a non-human recipient mammal; and (c) allowing the embryo to develop into at least one viable mammal in which the expression of said target gene is inhibited by the presence of the double-stranded target gene transcript.

In a related embodiment, the invention provides a method of producing a non-human mammal in which the expression of a target gene is inhibited by (a) providing a mammalian pre-implantation embryo, (b) removing the zona pellucid of the mammalian pre-implantation embryo, (c) providing a lentiviral vector as described herein, and (d) contacting the mammalian pre-implantation embryo with the lentiviral vector under conditions which permit the infection of the pre-implantation embryo to provide an infected pre-implantation embryo, (e) transfering the infected pre-implantation embryo into a non-human recipient mammal; and (f) allowing the embryo to develop into at least one viable mammal in which the expression of the target gene is inhibited by the presence of the double-stranded target gene transcript.

A transgenic non-human mammal in which the expression of a target gene is inhibited can be prepared in a number of ways. In order to achieve stable inheritance of the extra or exogenous siRNA transcript, the integration event must occur in a cell type that can give rise to functional germ cells, either sperm or oocyte. As described in further detail below, two animal cell types that can form germ cells and into which DNA can be introduced readily are fertilized egg cells and embryonic stem cells. Embryonic stem (ES) cells can be returned from in vitro culture to a host embryo where they become incorporated into the developing animal and can give rise to transgenic cells in all tissues, including germ cells. The embryonic stem cells are transducted in culture and the siRNA transgene is transmitted into the germline by injecting the cells into an embryo. The animals carrying mutated germ cells are then bred to produce a transgenic non-human mammal in which the expression of a target gene is inhibited. Expression of transgenes introduced by lentiviral vectors into murine and human embryonic stem cells has been described by Pfeiffer et al., Procl. Natl. Acad. Sci. USA 99:2140-2145 (2002), which is incorporated herein by reference.

The animals used as a source of fertilized eggs cells or embryonic stem cells can be any animal, although generally the preferred host animal is one which lends itself to multigenerational studies. Of particular interest are rodents including mice, such as mice of the FVB strain and crossed commercially available strains such as the (C57BL/6)×(BALB/c) hybrid, the (C57BL/6)×(SJL.F1) hybrid and the (SwissWebster)×(C57BL/6/DBA-z.F1) hybrid. The latter parental line also is referred to as C57B 16/D2. Other strains and cross-strains of animals can be evaluated using the techniques described herein for suitability for use in evaluating the inhibition of a target gene, for example, a goat, sheep, pig, cow or other domestic farm animal. In some instances, a primate, for example, a rhesus monkey can be desirable as the host animal, particularly for therapeutic testing.

One method for making transgenic non-human mammal in which the expression of a target gene is inhibited is by injection of a transducted embryonic stem cell into a pre-implantation embryo, for example, a morula or blastocyst. The method involves injecting the embryonic stem cell into a fertilized egg, or zygote, for example, at the blastocyst stage, and then allowing the egg to develop in a pseudo-pregnant mother. In this method of making a transgenic non-human mammal the transduction of embryonic stem cells by a lentiviral vector of the invention capable of inhibiting the expression of a target gene can lead to germ-line transmission of the target gene transcript.

As described herein, introducing a lentiviral vector of the invention can be introduced into the male pronucleus of a fertilized oocyte. The zygotes can either be transferred the same day, or cultured overnight to form 2-cell embryos that subsequently are implanted into the oviducts of pseudo-pregnant females. The offspring is subsequently screened for the presence of the target gene transcript. A pseudo-pregnant female is a female in estrous who has mated with a vasectomized male; she is competent to receive embryos but does not contain any fertilized eggs. Pseudo-pregnant females can serve as the surrogate mothers for embryos or embryonic stem cells transducted with a lentiviral vector of the invention.

Alternatively, a transgenic non-human mammal in which the expression of a target gene is inhibited can be prepared by directly transducing a pre-implantation embryo, for example, a morula or blastocyst, with a lentiviral vector of the invention. To achieve efficient transduction of the pre-implantation embryo it is desirable to remove the zona pellucid, a layer of extracellular matrix synthesized by the growing oocyte. Once transducted, the pre-implantation embryos that express the siRNA nucleic acid transcript can be selected, using a selectable marker system as described herein and vector-transduced embryos can be transferred into a pseudopregnant female. The resulting offspring can be analyzed for expression of the siRNA nucleic acid transcript and inhibition of target gene expression can be evaluated. Methods for transfer of a lentiviral vector into a pre-implantation embryo are known in the art and can be performed as described, for example, by Pfeiffer et al., supra, 2002, which is incorporated herein by reference in its entirety. Methods for generating transgenic animals, particularly animals such as mice, have become conventional in the art and are described, for example, in U.S. Pat. Nos. 4,736,866 and 4,870,009 and Hogan, B. et al., (1986) A Laboratory Manual, Cold Spring Harbor, N.Y., Cold Spring Harbor Laboratory.

Embryonic stem cells are derived from early mammalian embryos and display characteristics of totipotency, such that subsequent to being transferred to a suitable in vivo environment these cells contribute to the primary germ layers, ectoderm, endoderm, and mesoderm, and populate the germline of mice as described by Evans and Kaufman, Nature 292, 154-156 (1981) and Martin, Proc. Natl. Acad. Sci. USA 78, 7634-7638 (1981), both of which are incorporated herein by reference. Embryonic stem cells can be propagated in an undifferentiated state and genetically manipulated in vitro. A transgenic non-human mammal in which the expression of a target gene is inhibited can be generated by introducing a lentiviral vector of the invention into embryonic stem cells, followed by transplantation of the embryonic stem cells into embryos thereby effecting germ-line transmission. Methods of lentivectors transgenesis of embryonic stem cells are described in by Pfeiffer, supra, 2002.

An embryonic stem cell of the invention that has been transducted with a lentiviral vector can be stably propagated through undifferentiated proliferation. An embryonic stem cell of the invention further can be isolated from a cell line or derived directly from an embryo prior to transduction with the invention vector carrying the siRNA transgene. In vitro differentiation of the embryonic stem cells can be studied by culturing of embryonic stem cells in aggregates that form embroil bodies. The methods provided by the invention allow for stable expression of a lentiviral vector expressing a siRNA transgene formed by a nucleic acid sequence derived from a target gene transcript its reverse complement and, consequently, provide the capability of inhibiting target gene expression.

An embryonic stem cell of the invention that has been transducted with a lentiviral vector capable of inhibiting the expression of a target gene can be cultivated in hanging drops for a time appropriate to allow formation of an embryonic body. The inhibition of expression of a target gene can be evaluated in cells isolated from the embroil body. In one embodiment of the invention, a non-human embryonic stem cell infected with a lentivirus of the invention capable of inhibiting the expression of a target genes is injected into a non-human mammal to derive a tissue consisting of cells in which the target gene is inhibited. For example, a triatoma can be induced by injecting a suspension non-human embryonic stem cells into a host mammal, for example, a mouse, rat, dog, cow or monkey. Upon tumor formation fragments of the tissue can be removed and evaluated for the effects of target gene inhibition. The lentiviral vector and production system provided by the invention allow for transduction of embryonic stem cells that results in the stable expression of an siRNA capable of inhibiting target gene expression. The embryonic stem cells transducted via the invention methods can participate in formation of all three germ layers and stably express the siRNA transgene during differentiation, allowing for sustained target gene inhibition via the invention method. If desired, a stable cell line can be established from a cell isolated from a tissue that is derived from an embryonic stem cell infected with a lentiviral vector of the invention.

The invention, in a further embodiment, provides a method of selecting a compound that potentially reduces or eliminates a condition associated with a decrease in expression of a gene product by administering an agent to a transgenic non-human mammal in which the expression of a target gene is inhibited and determining whether the compound reduces or eliminates said condition.

The term “agent” is used herein to denote a chemical compound, a mixture of chemical compounds, a biological macromolecule, or an extract made from biological materials such as bacteria, plants, fungi, or animal (particularly mammalian) cells or tissues. Agents are evaluated for potential biological activity by inclusion in screening assays.

Numerous embodiments for the method described above are included within the scope of the invention. For example, a method for screening an agent for the ability to restore or modulate the effect of target gene inhibition by adding an agent to an appropriate cell line or introducing the agent into a transgenic non-human mammal or into a cell line in which the expression of a target gene is inhibited. Transgenic animals in which the expression of a target gene is inhibited as well as cell lines generated according to this invention can be used in these methods.

Generally a plurality of assay mixtures are run in parallel with different agent concentrations to obtain a differential response to the various concentrations. Typically, one of these concentrations serves as a negative control, i.e. at zero concentration or below the level of detection. Agents can be obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of naturally-occurring agents in the form of bacterial, fungal, plant and animal extracts are available or readily produced. Additionally, natural or synthetically produced libraries and compounds are readily modified through conventional chemical, physical and biochemical means, and can be used to produce combinatorial libraries. Known pharmacological agents can be subjected to directed or random chemical modifications, such as acylation, alkylation, esterification, amidification, to produce structural analogs.

An lentiviral vector of the invention capable of inhibiting the expression of at least one target gene also is useful in therapeutic applications designed to inactivate disease-associated transcripts and thereby reduce the severity of inherited metabolic, infectious or malignant conditions. The therapeutic applications of the invention can be used to reduce the severity of dominant genetic conditions, including those caused by a point mutation, by inhibiting the expression of the mutant allele while leaving unaffected the expression of the remaining wild-type transcript. This embodiment of the invention capitalizes on the sequence specificity of siRNA which requires perfect match for target gene inhibition. Any condition that can be reduced in severity by decreasing the expression of a gene product can be appropriate for the screening and therapeutic methods of the invention including, for example, cancer, hemophilia, diabetes, Alzheimer's disease as well as triplet repeat expansion diseases including fragile X syndrome, Huntington's chorea, myotonic muscular dystrophy, spinocerebellar atrophy, Friedreich ataxia, dentatorubral and pallidoluysian atrophy, and Machado-Joseph disease.

For ex vivo therapy applications using lentiviral vectors of the invention, cells are removed from a subject and cultured in vitro. The siRNA transcript is introduced into the cells in vitro via transduction with a lentiviral vector of the invention and subsequently the modified cells are expanded in culture followed by reimplantation into the subject. Methods for lentiviral gene transfer via transduction, which are described herein and known in the art, allow for the transfer into and subsequent stable expression of siRNA target gene transcripts by somatic cells. Ex vivo applications can involve, for example, partial hepatectomy and isolation of hepatocytes from an individual with defective gene function, transduction of the lentiviral vector, and finally transplantation of the transducted cells.

The therapeutic applications of the present invention include delivery of the lentiviral vectors of the invention into somatic, nonreproductive cells as well as into reproductive, germ line cells of host mammals. Mammals carrying foreign exogenous genes in their germ line, generally referred to as transgenic animals, presently include, for example, mice, rats, rabbits, and some domestic livestock.

For in vivo gene therapy, using lentiviral vectors of the invention capable of inhibiting a target gene via expression of a siRNA target gene transcript, cells to be transducted are not removed from the subject. Rather, the siRNA transgene is introduced into cells of the recipient organism in situ that is, within the recipient. In vivo gene therapy has been reported in several animal models and the methods described herein are specifically contemplated for human gene therapy. For a description of viral vectors and their uses in gene therapy, see, for example, Gene Therapy: Principles and Applications (T. Blankenstein, et., 1999, Springer-Verlag, Inc.) and Understanding Gene Therapy (N. Lemoine, ed., 2000, R-G Vile), both of which are incorporated herein in their entirety.

Furthermore, in vivo applications encompass transduction of a mammalian cell in utero, more specifically, into the somatic cells of a mid-trimester fetus. The rationale for human in utero gene therapy is that it allows the correction of some types of genetic diseases before the appearance of any clinical manifestations; in addition, introduction of a therapeutic lentivectors into the fetus offers a number of potential advantages over postnatal gene transfer. For neurologic genetic diseases that appear to produce irreversible damage during gestation, treatment before birth, if desired early in pregnancy, can be useful to allow the birth of a normal baby. The lentiviral vectors of the invention integrate efficiently into the target cell's genome and therefore insert the therapeutic nucleic acid sequence permanently into the genetic make-up of the cell. For genetic diseases that can be treated or reduced in severity by inhibiting a target gene product and where correction for the lifetime of the individual is desired, the lentiviral vectors provided by the invention are particularly useful. Successful early treatment with a lentiviral vector of the invention can preempt the appearance of any clinical manifestations of a disease. Furthermore, gene transfer in the fetus can be more efficient than in the more mature organism, so that gene therapy should be easier to accomplish prenatally than postnatally. In addition, the immunological naivete and the permissive environment of the early gestational fetus allow acceptance of cells and lentivectors without the need for immunosuppression or myeloablation because during early immunologic development, before thymic processing of mature lymphocytes, the fetus is largely tolerant of foreign antigens.

The pharmaceutically acceptable vehicle for a therapeutic lentiviral vector can be selected from known pharmaceutically acceptable vehicles, and should be one in which the virus is stable. For example, it can be a diluent, solvent, buffer, and/or preservative. An example of a pharmaceutically acceptable vehicle is phosphate buffer containing NaCl. Other pharmaceutically acceptable vehicles, for example, aqueous solutions, non-toxic excipients, including salts, preservatives, buffers and equivalents are described in Remington's Pharmaceutical Sciences, 15th Ed. Easton: Mack Publishing Co. pp 1405-1412 and 1461-1487 (1975) and The National Formulary XIV, 14th Ed. Washington: American Pharmaceutical Association (1975), the contents of which are hereby incorporated by reference.

If desired, the lentiviral vector of the invention can be introduced into the cell by administering the lentiviral vector of the invention to a mammal that carries the cell. For example, the lentiviral vector of the invention can be administered to a mammal by subcutaneous, intravascular, or intraperitoneal injection. If desired, a slow-release device, such as an implantable pump, can be used to facilitate delivery of the lentiviral vector of the invention to cells of the mammal. A particular cell type within a mammal can be targeted by modulating the amount of the lentiviral vector of the invention administered to the mammal and by controlling the method of delivery. For example, intravascular administration of the lentiviral vector of the invention to the portal, splenic, or mesenteric veins or to the hepatic artery can be used to facilitate targeting the lentiviral vector of the invention to liver cells. In another method, the lentiviral vector of the invention can be administered to cells or organ of a donor individual (human or non-human) prior to transplantation of the cells or organ to a recipient.

In a preferred method of administration, the lentiviral vector of the invention is administered to a tissue or organ containing the targeted cells of the mammal. Such administration can be accomplished by injecting a solution containing the lentiviral vector of the invention into a tissue, such as skin, brain (e.g., the olfactory bulb), kidney, bladder, trachea, liver, spleen, muscle, thyroid, thymus, lung, or colon tissue. Alternatively, or in addition, administration can be accomplished by perfusing an organ with a solution containing the lentiviral vector of the invention, according to conventional perfusion protocols.

In another therapeutic embodiment, the lentiviral vector of the invention is administered intranasally by applying a solution of the lentiviral vector of the invention to the nasal mucosa of a mammal. This method of administration can be used to facilitate transportation of the lentiviral vector of the invention into the brain. This delivery mode provides a means for delivering the lentiviral vector of the invention to brain cells, in particular, mitral and granule neuronal cells of the olfactory bulb, without subjecting the mammal to surgery. In an alternative method for using the lentiviral vector of the invention to express an siRNA transgene in the brain, the lentiviral vector of the invention is delivered to the brain by osmotic shock according to conventional methods for inducing osmotic shock.

Although described for lentiviral vectors and corresponding production system, the invention also can be practiced with equivalents including other viral based systems able to introduce relatively high levels of nucleic acid sequences into a variety of cells. Suitable viral vectors include yet are not limited to Herpes simplex virus vectors (Geller et al., Science 241:1667-1669 (1988)); vaccinia virus vectors (Piccini et al., Meth. Enzymology 153:545-563 (1987)); cytomegalovirus vectors (Mocarski et al., in Viral Vectors, Y. Gluzman and S. H. Hughes, Eds., Cold Spring Harbor Laboratory, Cold Spring Harbor, N.Y., 1988, pp. 78-84)); Moloney murine leukemia virus vectors (Danos et al., Proc. Natl. Acad. Sci. USA 85:6460-6464 (1988); Blaese et al., Science 270:475-479 (1995); Onodera et al., J. Viol. 72:1769-1774 (1998)); adenovirus vectors (Berkner, Biotechniques 6:616-626 (1988); Cotten et al., Proc. Natl. Acad. Sci., USA 89:6094-6098 (1992); Graham et al., Meth. Mol. Biol. 7:109-127 (1991); Li et al., Human Gene Therapy 4:403-409 (1993); Zabner et al., Nature Genetics 6:75-83 (1994)); adeno-associated virus vectors (Goldman et al., Human Gene Therapy 10:2261-2268 (1997); Greelish et al., Nature Med. 5:439-443 (1999); Wang et al., Proc. Natl. Acad. Sci., USA 96:3906-3910 (1999); Snyder et al., Nature Med. 5:64-70 (1999); Herzog et al., Nature Med. 5:56-63 (1999)); retrovirus vectors (Donahue et al., Nature Med. 4:181-186 (1998); Shackleford et al., Proc. Natl. Acad. Sci. USA 85:9655-9659 (1988); U.S. Pat. Nos. 4,405,712, 4,650,764 and 5,252,479, and WIPO publications WO 92/07573, WO 90/06997, WO 89/05345, WO 92/05266 and WO 92/14829. The skilled person understands that these and other methodologies can be useful equivalents for practicing the invention.

It is understood that modifications which do not substantially affect the activity of the various embodiments of this invention are also included within the definition of the invention provided herein. Accordingly, the following examples are intended to illustrate but not limit the present invention.

### EXAMPLE I

### Inhibition of Target Gene Expression in Mammalian Cells

This example demonstrates the ability of a lentivector containing nucleic acid sequences that form a double-stranded target gene transcript corresponding to an siRNA to inhibit the expression of a target gene in a variety of cell types.

A lentiviral vector expressing GFP driven by either a cytomegalovirus (CMV) immediate early promoter or a cytomegalovirus enhancer/chicken β-actin (CAG) promoter was used to transduct 293T cells either alone or by co-transduction with a lentiviral vector expressing siRNA specific for GFP. LV-green fluorescent protein (GFP) was constructed by cloning the CAG promoter into the ClaI and BamHI sites of the vector LV-pGFP (see, Follenzi et al., Nat. Genet. 25, 217-222 (2000), which is incorporated herein by reference), thereby replacing the phosphoglycerate kinase (PGK) promoter. LV-Lac was cloned by introducing the LacZ-woodchuck hepatitis virus fragment into the NheI and KpnI sites of LV-GFP, thereby replacing the enhanced GFP (eGFP) cassette with LacZ. The lentiviral vectors were produced as described in Pfeifer et al., supra, 2000 and Dull et al., supra, 1998, both of which are incorporated herein by reference.

Briefly, the lentiviral vectors were produced by using a four-plasmid, third-generation, Tat-free packaging system as described. The two packaging plasmids (encoding HIV gag, pol, and rev), together with the plasmid coding for vesicular stomatitis virus envelope and the vector itself, were transfected into 293T cells by using the calcium phosphate method. Typically, 12 15-cm dishes were transfected and virus was harvested by collecting the cell culture medium 24, 48, and 72 h after changing the transfection medium to DMEM containing 10% FCS. After filtering the collected medium through 0.45-μm filters, the virus was concentrated by spinning at 68,000×g for 2 h, followed by a second spin (59,000×g for 2.5 h at room temperature). The resulting pellet was resuspended in 200 μl of Hanks' buffer. The titer of lentiviral vectors was determined by measuring the amount of HIV p24 gag antigen by ELISA (Alliance; NEN). To calculate the amount of infectious units (I.U.), the p24 titer was correlated to the biological activity of a similar virus carrying a green fluorescent protein (GFP) cassette by using serial dilutions of the GFP virus to transduce 293T cells (1 ng of p24=1×105 I.U.).

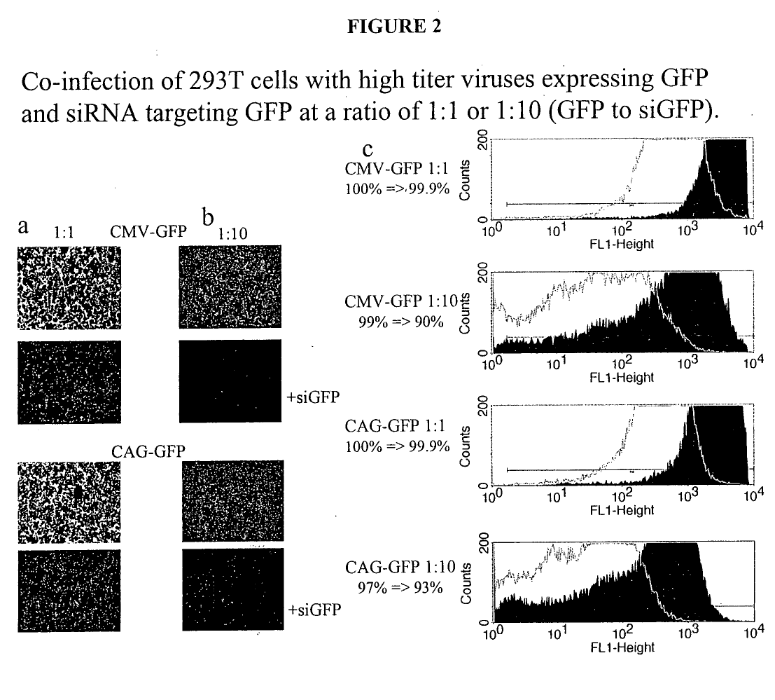

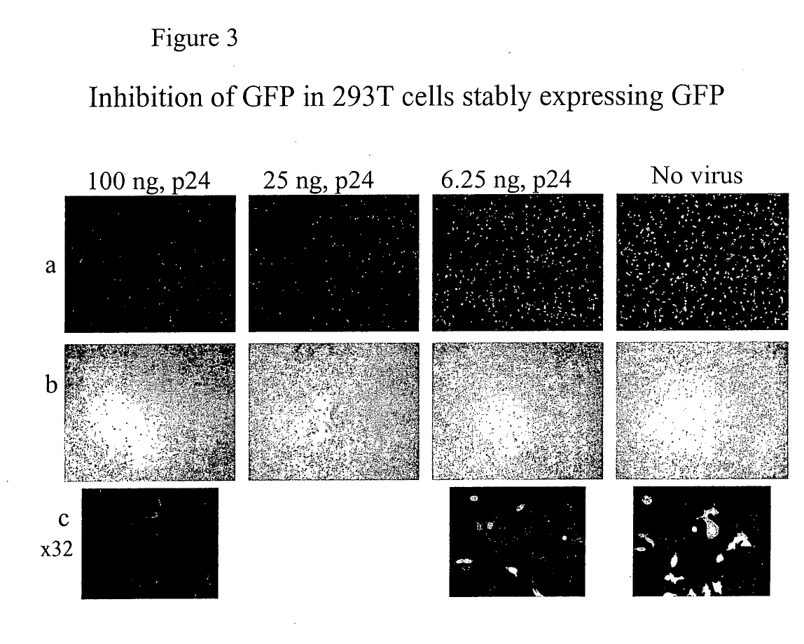

Two sets of 293T cells were transduced, one set with a 1:1 ratio of GFP to siGFP, the second set with a 1:10 ratio of GFP to siGFP. As shown in FIG. 2, siGFP inhibited expression of the target gene at both ratios and with both promoter systems. 293T cells stably expressing GFP also were transducted with siGFP lentivirus. FIG. 3 shows inhibition of GFP in 293T cells infected with 100 ηg, 25 ηg and 6.25 ηg of p24 virus and uninfected cells visualized by fluorescence microscopy at ×5 magnification, ×32 magnification and by light microscopy at ×5 magnification.

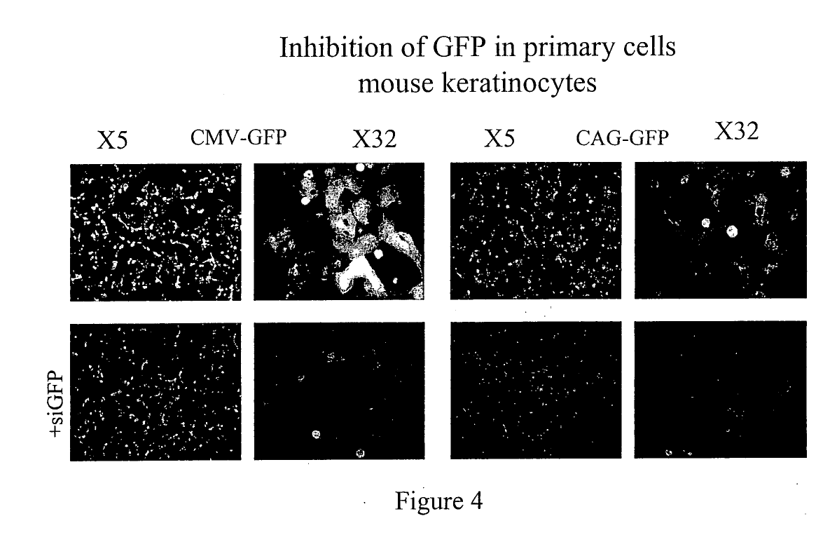

Mouse primary skin keratinocytes were transducted with lentivirus expressing GFP from either CMV or a CAG promoter, either alone or by co-transduction with siGFP. The cells were transduced either with 100 ηg of p24 of GFP virus alone or were co-transduced with 100 ηg of p24 of siGFP. FIG. 4 shows inhibition of GFP in primary mouse keratinocytes transducted with lentivirus expressing GFP from either CMV or a CAG promoter, either alone or by co-transduction with siGFP.

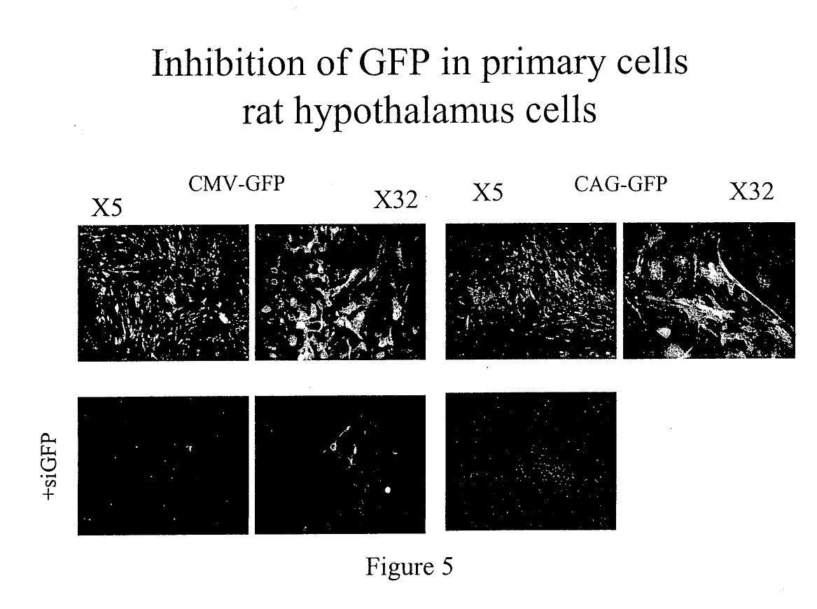

Rat brain primary hypothalamus cells were transduced with lentivirus expressing GFP from either CMV or a CAG promoter, either alone or by co-transduction with siGFP. The cells were injected either with 100 ηg of p24 of GFP virus alone or were co-transduced with 100 ηg of p24 of siGFP. FIG. 5 shows target gene inhibition of the GFP gene by siGFP.

To generate transgenic mice, six-week-old CB6 μl (C57BL/6×BALB/c) females were superovulated with 5 units of pregnant mare serum gonadotropin (Sigma), followed 48 h later by injection of 5 units of human gonadotropin (Sigma), and mated with CB6 μl males. Morulae (8-16-cell embryos) were isolated by flushing the oviduct 2.5 days postcoitus with M2 medium (Sigma). Removal of the zona pellucida was achieved by acidic tyrode treatment as described in Manipulating the Mouse Embryo, (eds. Hogan, B., Beddington, R., Costantint, F. & Lacy, E.; Cold Spring Harbor Lab. Press, Plainview, N.Y., 1994), which is incorporated herein in its entirety. Morulae were transduced overnight with 20 ng of P24/ml in a volume of 5 μl, covered with light paraffin oil (Fisher). Thirty hours after transduction, blastocysts were transferred into the uteri of pseudopregnant CB6F1 mice. The presence of the lentiviral vector DNA was detected by PCR, using primers (5′-CAAGGCAGCTGTAGATCTTAGCC-3′ and 5′-GATCTTGTCTTCGTTGGGAGTG-3′) that amplify a 300-bp fragment of the siGFP cassette.

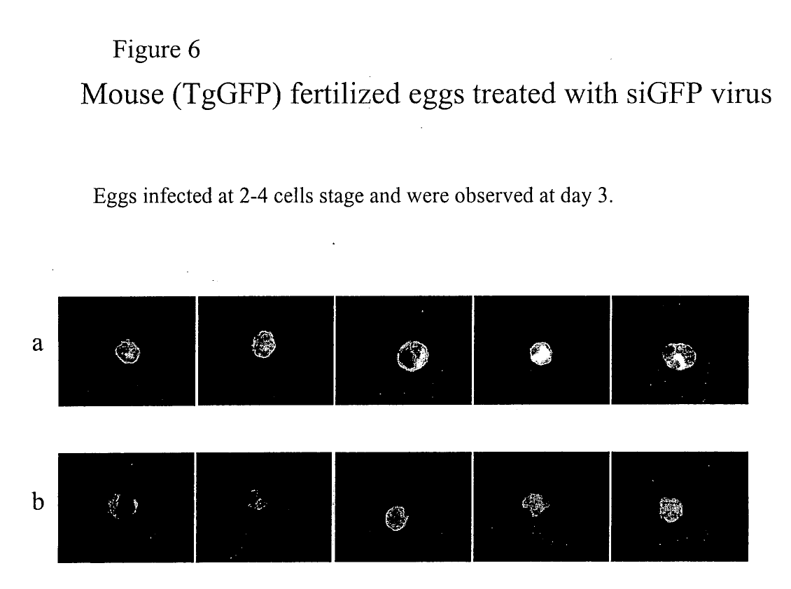

Fertilized mouse eggs were subsequently isolated from mice transgenic for GFP and treated with siGFP in culture. FIG. 6 shows (a) siGFP treated eggs visualized by fluorescence microscopy at ×32 magnification, and (b) untreated eggs visualized by fluorescence microscopy at ×32 magnification.

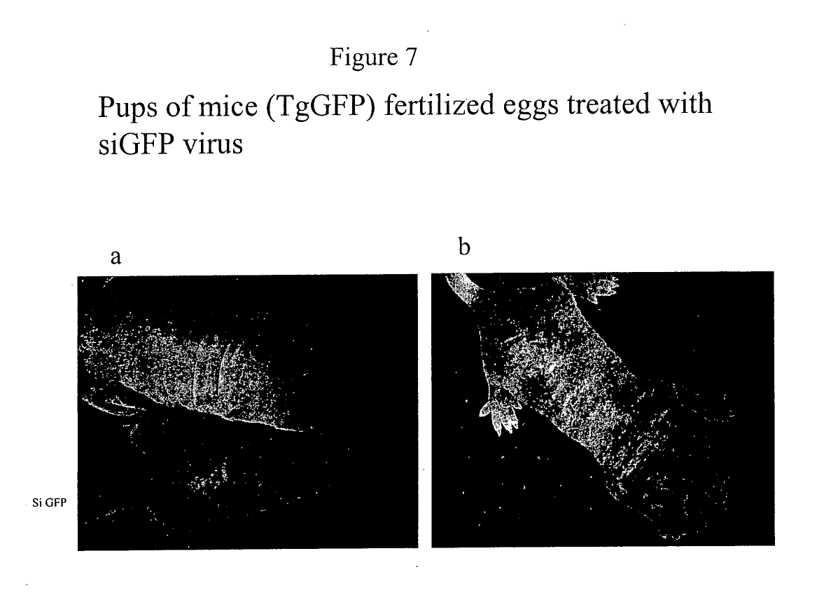

To analyze gene inhibition during embryogenesis in vivo, fertilized siGFP treated mouse eggs were implanted into pseudopregnant female mice. FIG. 7 shows (a) an siGFP affected pup compared to an unaffected pup, and (b) an affected pup showing a patchy chimera pattern of GFP expression demonstrating that lentiviral genes are expressed even during in vivo development of the mouse embryo.

Hela cells also were transducted with a lentivirus containing siGFP in forward orientation, sip53, parental vector without insert, and siGFP in reverse orientation. All siRNA inserts were driven by the human polIII H1 promoter and all vectors contained the reporter gene encoding for the green fluorescent protein (GFP) under the control of the cytomegalovirus (CMV) promoter (GFP-CMV). The hH1 siRNA is cloned into the the 3′LTR in these vectors such that the cassette is duplicated upon integration, yielding a ratio of siRNA to target gene of 2:1 for GFP and 1:1 for sip53. Both siRNAs inhibited target gene expression compared to the parental vector.

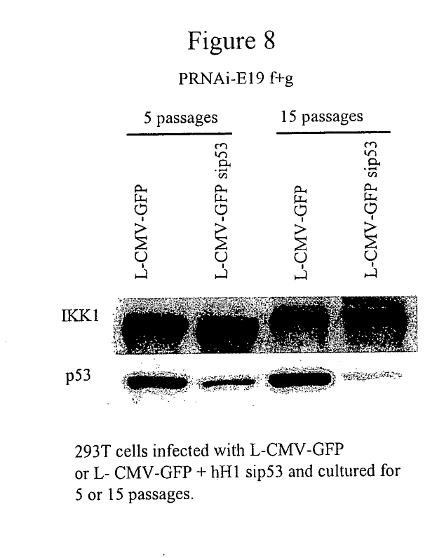

293 T cells also were transducted with a lentiviral vector carrying GFP-CMV either with (L-CMV-GFP-hH1 sip53) or without (L-CMV-GFP) an hH1sip53 cassette insert. Due to the presence of the T-antigen, p53 is stable in 293T cells. Consequently, to show inhibition of p53 target gene expression, the cells were cultured for 5 to 15 passages to allow for dilution of the originally present p53 protein. FIG. 8 shows specific gene inhibition of p53 by a lentivector carrying p53 siRNA.

### EXAMPLE II

### Creation of an Inducible siRNA Lentiviral Vector Using a Cre-LoxP System

This example demonstrates preparation of an inducible siRNA lentiviral vector using the Cre-LoxP system.

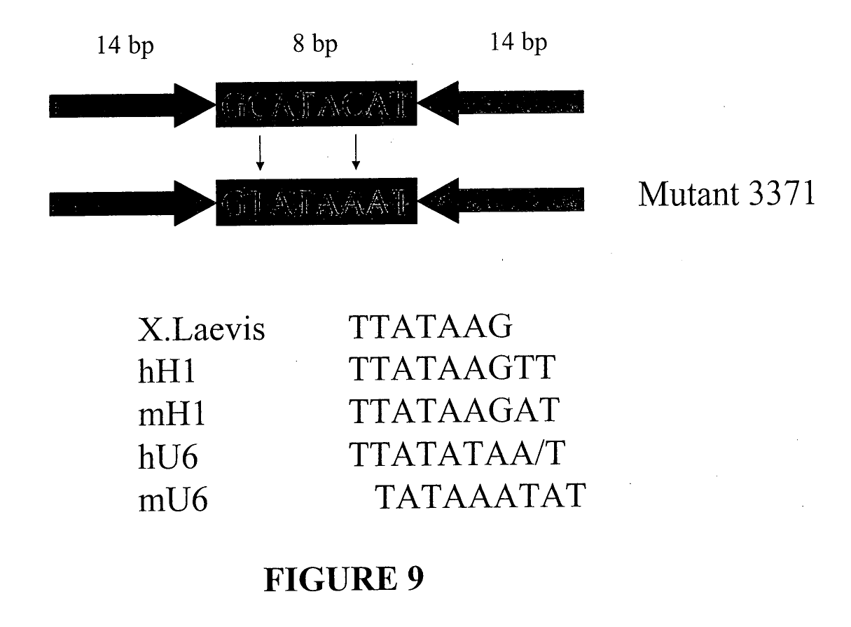

Briefly, Cre is a 38 kDa recombinase protein f rom bacteriophage P1 which mediates intramolecular (excisive or inversional) and intermolecular (integrative) site specific recombination between loxP sites. A loxP site consists of two 14 base pair inverted repeats separated by an 8 base pair asymmetric spacer region (FIG. 9). One molecule of Cre binds per inverted repeat or two Cre molecules line up at one loxP site. The recombination occurs in the asymmetric spacer region. The 8 base pairs of the asymmetric spacer region also are responsible for the directionality of the site such that loxP sequences in opposite orientation to each other invert the intervening piece of DNA, while two sites in direct orientation dictate excision of the intervening DNA between the sites leaving one loxP site behind.

The precise removal of a nucleic acid sequence is used to make a silencing cassette that is inactive until CRE recombinase is expressed. To this end, a stuffer fragment is inserted between the mU6 promoter and the siRNA hairpin. The presence of the stuffer impedes transcription of the siRNA hairpin due to the presence of polIII termination signals in the stuffer. The stuffer fragment is flanked by loxP sites. Upon CRE expression, the stuffer fragment is recombined out, leaving a single copy of the loxP site and resulting in juxtaposition of the mU6 promoter with the siRNA hairpin. The result is transcription of the hairpin and silencing of the target gene.

PolIII promoters are compact and sensitive to alteration of the spacing between the three different promoter elements, which are a distal sequence element (DSE), a proximal sequence element (PSE) and a TATA box. An ideal position for a loxP site can be either between the PSE and the TATA BOX or between the TATA box and the transcriptional start site. However, the distance between the PSE and the TATA box is ˜20 bp, as well as the distance between the TATA box and the transcriptional starte site is also ˜20 bp such that it is not possible to insert the complete loxP site, which consists of 34 bp, in either of these locations. To find a position between the mU6 promoter and the siRNA hairpin in which a single loxP site can be inserted without affecting the transcriptional capability of the promoter, so that the ‘ON’ configuration results in efficient transcription of the siRNA hairpin and thus efficient silencing, the fact that efficient recombination depends on the 14 base pair direct repeat sequences and, to a lesser extent, on the sequence of the 8 base pair linker was taken into account. As shown in FIG. 9, the 8 base pair linker sequence designated Mutant 3371 differs by only two nucleotides from the mU6 TATA box sequence as described by Lee and Saito, Gene 216 (1):55-65 (1998), which is incorporated herein by reference.

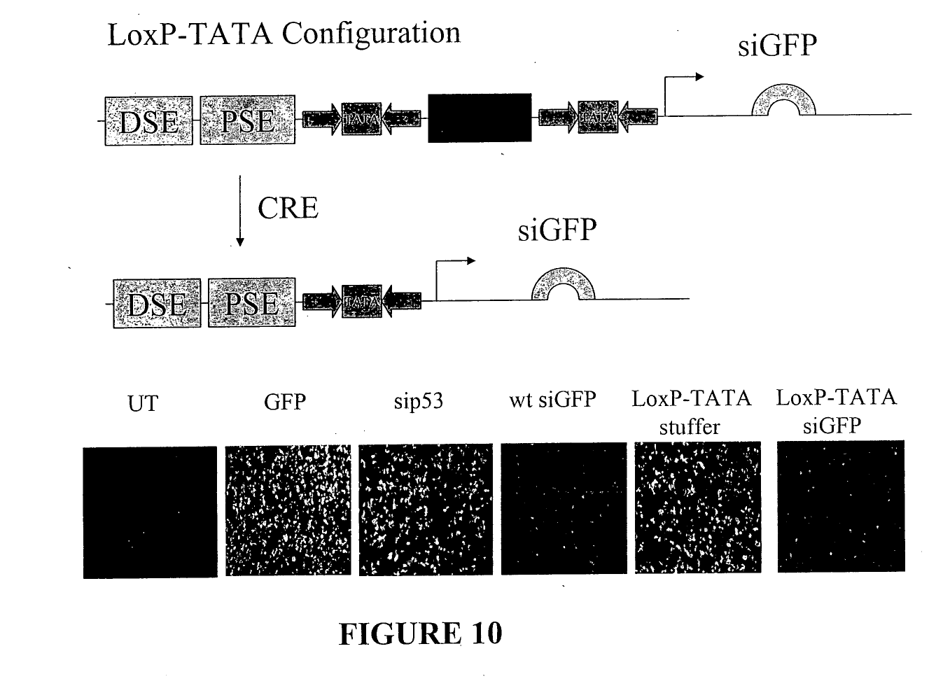

Briefly, a loxP site containing a linker with the two mutations required to constitute a mU6 TATA box, referred to as loxP TATA, is competent for CRE mediated recombination, albeit at a lower efficiency than the wt loxP sequence. An mU6 promoter was constructed in which the loxP TATA is cloned into the region between the PSE and the transcriptional start site. The resulting construct consists of a mU6 promoter, a loxP TATA flanked stuffer and an siRNA hairpin that is inactive and thus incapable of silencing its target (FIG. 10).

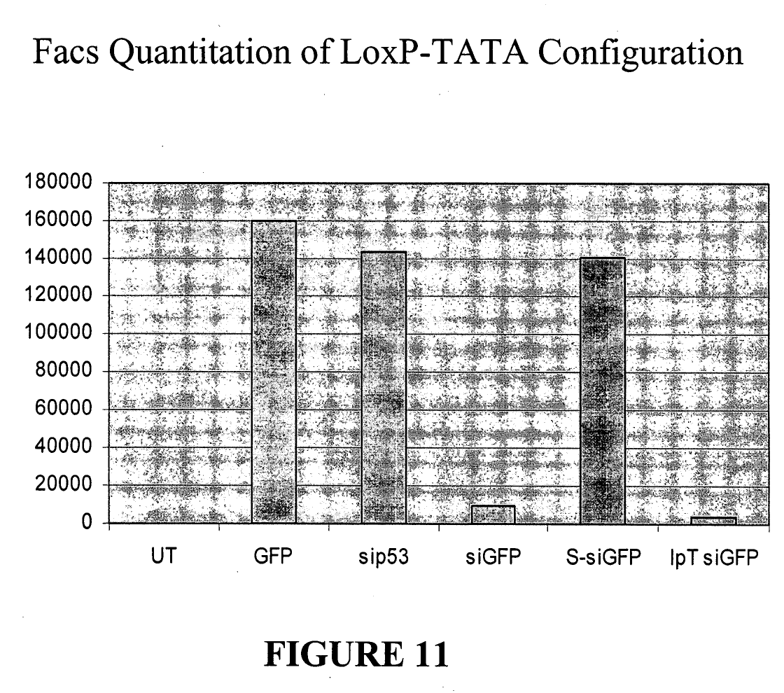

To test whether the ‘OFF’ construct was in fact inactive and whether the ON contruct, containing one loxP TATA site as is present upon administration of CRE, was effective in target gene inhibition, constructs corresponding to the “ON” and ‘OFF’ position were prepared and tested for activity. As shown in FIG. 10 with GFP as the target, in the ‘OFF’ configuration, the construct (LoxP-TATA stuffer) consisting of a mU6 promoter, a loxP TATA flanked stuffer and an siRNA hairpin was inactive and incapable of silencing its target. Conversely, the construct (LoxP-TATA siGFP) consisting of a mU6 promoter, one loxpTATA site and an siRNA hairpin, which corresponds to the ‘ON’ configuration, was active and capable of silencing its target. While distinct constructs corresponding to the ‘ON’ and ‘OFF’ positions were prepared and tested without administration of recombinase, delivery of CRE recombinase, induces a single construct from the ‘OFF’ configuration to the ‘ON’ configuration and triggers silencing of the GFP target. FIG. 11 shows quantitation by FACS analysis of GFP levels in the presence of the different constructs, in particular, GFP target inhibition with the LoxP-TATA siGFP (IpT siGFP) construct compared to lack of target inhibition by the LoxP-TATA stuffer (S-siGFP) construct.

The construct was subsequently transferred into a lentiviral vector of the invention for induction of inhibition of target gene expression by contacting the lentiviral vector with CRE recombinase.

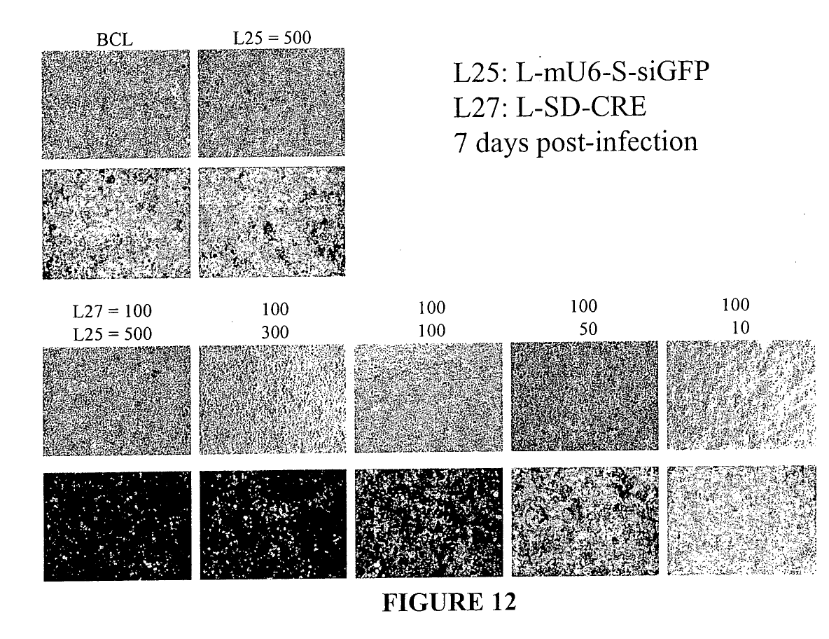

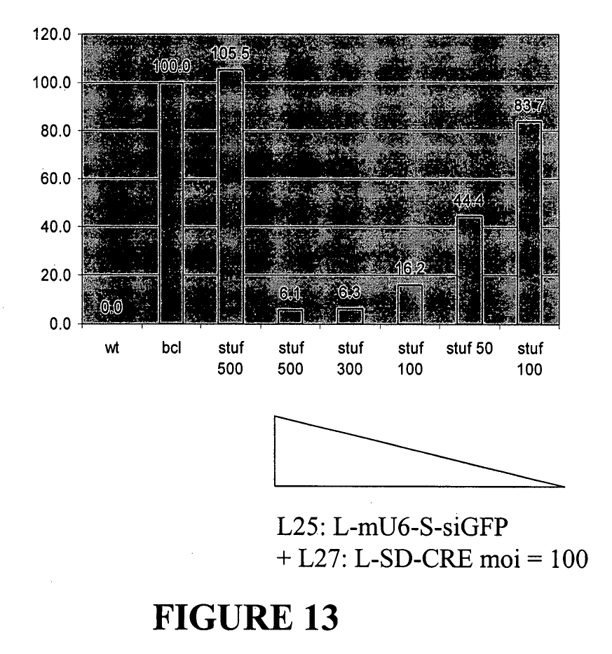

FIG. 12 shows the effects of transducing 293T cells, which stably express GFP, with two lentiviral vectors, L25 and L27. The L25 vector carries a silencing cassette against GFP in the OFF configuration, in particular, a mU6 promoter, a loxP flanked stuffer and a siRNA against GFP. The L27 vector expresses CRE recombinase. GFP positive cells were transduced with decreasing amounts of L25 and a fixed amount of L27. Eight days after infection, GFP levels were quantitated by FACS analysis and shown to be inversely correlated with the amount of L25 (FIG. 13).

The results demonstrate the capability of an inducible lentiviral vector of the invention to downregulate specific genes in a tissue specific manner by putting CRE under the control of a tissue specific promoter. Furthermore, using the lentiviral delivery system, the target gene can be downregulated in specific regions of a tissue.

### EXAMPLE III

### Expression of Multiple siRNA Transcripts from a Lentivirus Vector

A lentiviral vector was prepared using multiple cassettes expressing different siRNA's. This allows for inhibition of multiple target nucleic acid sequences simultaneously using one lentivirus vector. The number of different siRNA transcripts that can be expressed simultaneously is limited only by the packaging capacity of lentiviral vector. In particular, two adjacent promoters driving distinct siRNA transcripts were found not to interfere with each other.

Throughout this application various publications have been referenced within parentheses. The disclosures of these publications in their entireties are hereby incorporated by reference in this application in order to more fully describe the state of the art to which this invention pertains.

Although the invention has been described with reference to the disclosed embodiments, those skilled in the art will readily appreciate that the specific experiments detailed are only illustrative of the invention. It should be understood that various modifications can be made without departing from the spirit of the invention. Accordingly, the invention is limited only by the following claims.

We claim:

1. A lentiviral vector capable of inhibiting the expression of a target gene comprising a first nucleic acid sequence derived from a target gene transcript and a second nucleic acid sequence corresponding to the reverse complement of said first nucleic acid sequence.

2. The lentiviral vector of claim 1, wherein said first and said second nucleic acid sequences are each between 19 and 22 nucleotides in length.

3. The lentiviral vector of claim 1, wherein transcription of said first and said second nucleic acid sequences are driven by a single promoter.

4. The lentiviral vector of claim 3, wherein said promoter is capable of mammalian expression.

5. The lentiviral vector of claim 4, wherein said first and said second nucleic acid sequences are separated by a spacer sequence.

6. The lentiviral vector of claim 5, wherein said first and said second nucleic acid sequences align to form a double-stranded transcript having a hairpin structure.

7. The lentiviral vector of claim 6, wherein said double-stranded transcript formed by first and said second nucleic acid sequences is capable of inhibiting the expression of said target gene.

8. The lentiviral vector of claim 7, further comprising sets of said first and said second nucleic acid sequences capable of forming more than one said double-stranded transcript.

9. The lentiviral vector of claim 8, wherein each of said double stranded transcripts is capable of inhibiting the expression of a distinct target gene.

10. The lentiviral vector of claim 7, wherein said lentivirus is selected from the group consisting of human immunodeficiency virus-1 (HIV-1), human immunodeficiency virus-2 (HIV-2), simian immunodeficiency virus (SIV), feline immunodeficiency virus (FIV) and equine infectious anemia virus (EIAV).

11. The lentiviral vector of claim 10, wherein said lentivirus is HIV-1.

12. The lentiviral vector of claim 4, wherein said promoter is a RNA polymerase III promoter.

13. The lentiviral vector of claim 12, wherein said promoter is of human origin.

14. The lentiviral vector of claim 12, wherein said promoter is of murine origin.

15. The lentiviral vector of claims 13 or 14, wherein said promoter is selected from the group consisting of H1RNA and U6.

16. The lentiviral vector of claim 15, further comprising nucleic acid sequences sufficient for induction by a site-specific recombinase.

17. The lentiviral vector of claim 16, wherein said recombinase is cre recombinase.

18. The lentiviral vector of claim 16, wherein said promoter is U6.

19. The lentiviral vector of claim 18, wherein said U6 promoter further comprises a stuffer nucleic acid sequence.

20. The lentiviral vector of claim 19, wherein said stuffer nucleic acid sequence is flanked by loxP sites.

21. The lentiviral vector of claim 20, wherein contact with cre recombinase intiates a recombination event that comprises excision of the stuffer nucleic acid.

22. The lentiviral vector of claim 20, wherein said recombination event further comprises juxtaposition of the promoter and the first and second nucleic acid sequences driven by said promoter.

23. The lentiviral vector of claim 22, wherein said recombination event results in transcription of said first and second nucleic acid sequences.

24. The lentiviral vector of claim 1, wherein said vector is non-replicating.

25. A mammalian cell stably transducted with the vector of claims 1 or 11.

26. The mammalian cell of claim 25, wherein said cell is a non-dividing cell.

27. The mammalian cell of claim 25, wherein said cell is transducted in vitro.

28. The mammalian cell of claim 25, wherein said cell is transducted in vivo.

29. A lentiviral vector production system comprising:
(a) a packaging component of lentiviral structural proteins; and
(b) a transfer vector component comprising lentiviral cis-acting nucleic acid sequences and further comprising a first nucleic acid sequence derived from a target gene transcript and a second nucleic acid sequence corresponding to the reverse complement of said first nucleic acid sequence, wherein said first and said second vector components are sufficient to produce a lentiviral vector capable of inhibiting the expression of said target gene in a cell.

30. The lentiviral vector production system of claim 29, wherein said vector production system further comprises an inducible promoter.

31. The lentiviral vector production system of claim 30, wherein said promoter is capable of mammalian expression.

32. The lentiviral vector production system of claim 29, wherein the lentivirus is HIV-1.

33. The lentiviral vector production system of claim 29, wherein said packaging component comprises more than one physically distinct nucleic acid molecule.

34. The lentiviral vector production system of claims 29, 31 or 32, further comprising a envelope pseudotype component comprising a nucleic acid sequence encoding a polypeptide that modulates the host range of said lentiviral vector.

35. A method of producing a pseudotyped lentiviral vector capable of inhibiting the expression of a target gene comprising transfecting a host cell with the lentiviral production system of claim 34.

36. A pseudotyped lentiviral vector produced by the method of 35.

37. The pseudotyped lentiviral vector of claim 36, wherein said vector is non-replicating.

38. A method of inhibiting the expression of a target gene in a cell comprising contacting a cell under conditions that permit infection with a lentiviral vector comprising a first nucleic acid sequence derived from a target gene transcript and a second nucleic acid sequence corresponding to the reverse complement of said first nucleic acid sequence under the control of at least one promoter, wherein upon transcription said nucleic acid sequences form a double-stranded target gene transcript that inhibits target gene expression.

39. The method of claim 38, wherein said lentiviral vector is derived from HIV-1.

40. The method of claim 38, further comprising more than one lentiviral vector.

41. The method of claim 38, wherein said lentiviral vector comprises nucleic acid sequences derived from more than one target gene.

42. The method of claims 40 or 41, wherein the expression of more than one target gene is inhibited in said cell.

43. The method of claim 38, wherein said promoter is capable of mammalian expression.

44. The method of claim 38, wherein said promoter is inducible.

45. The method of claim 38, wherein said cell is selected from the group consisting of 293T, primary skin keratinocyte and primary hypothalamus cell, non-human mammalian fertilized oocyte, and non-human mammalian embryonic stem cell.

46. The method of claim 45, wherein said cell is of murine, porcine, bovine or primate origin.

47. The method of claim 44, wherein said cell is contacted in utero.

48. The method of claim 45, wherein said cell is a non-human mammalian fertilized oocyte.

49. The method of claim 45, wherein said cell is a non-human mammalian embryonic stem cell.

50. The method of claim 49, further comprising injecting said non-human embryonic stem cell into a non-human mammal.

51. A cell isolated from a tissue that is derived from the embryonic stem cell of claim 49.

52. A cell line derived from the isolated cell of claim 51.

53. The method of claim 49, further comprising cultivating said embryonic stem cell under conditions which permit formation of embryoid bodies.

54. A method of producing a non-human mammal in which the expression of a target gene is inhibited, said method comprising the steps of:
(a) infecting a pre-implantation mammalian embryo with the lentiviral vector of claim 11;
(b) transferring said infected pre-implantation embryo into a non-human recipient mammal; and
(c) allowing said embryo to develop into at least one viable mammal in which the expression of said target gene is inhibited by the presence of said double-stranded target gene transcript.


SEQUENCE LISTING (9 sequences)

SEQ ID NO: 1 - DNA, 23 bp, Artificial Sequence
  Primer
caaggcagctgtagatcttagcc

SEQ ID NO: 2 - DNA, 22 bp, Artificial Sequence
  Primer
gatcttgtcttcgttgggagtg

SEQ ID NO: 3 - DNA, 8 bp, Artificial Sequence
  Synthetic
gcatacat

SEQ ID NO: 4 - DNA, 8 bp, Artificial Sequence
  Synthetic
gtataaat

SEQ ID NO: 5 - DNA, 7 bp, Xenopus laevis
ttataag

SEQ ID NO: 6 - DNA, 9 bp, Homo sapien
ttataagtt

SEQ ID NO: 7 - DNA, 9 bp, Mus muculus
ttataagat

SEQ ID NO: 8 - DNA, 8 bp, Homo sapiens
  misc_feature 8: N=A or T
ttatatan

SEQ ID NO: 9 - DNA, 9 bp, Mus muculus
tataaatat